(19) United States
(12) Patent Application Publication
(10) Pub. No.: US 2005/0227980 A1
(43) Pub. Date: Oct. 13, 2005

(54) Aralkyl and aralkylidene heterocyclic lactam and imides

(75) Inventors: Harry R. Howard JR., Bristol, CT (US); Mark Elliott, Canterbury, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/011,912

(22) Filed: Dec. 14, 2004

Related U.S. Application Data
(60) Provisional application No. 60/529,856, filed on Dec. 15, 2003.

(51) Int. Cl. C07D 413/02; A61K 31/5377

(52) U.S. Cl. 514/235.5; 544/121

(57) ABSTRACT

The present invention relates to compounds of the formula I 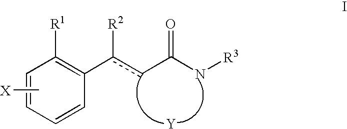
wherein R¹, R², R³, X, Y and the dashed line are as defined in the specification, to intermediates for their preparation, to pharmaceutical compositions containing them and to their medicinal use as psychotherapeutic agents.

### BACKGROUND OF THE INVENTION

The present invention relates to novel aralkyl and aralkylidene heterocyclic lactams and imides, to intermediates for their preparation, to pharmaceutical compositions containing them and to their medicinal use. The compounds of the present invention include selective agonists and antagonists of serotonin 1 (5-HT₁) receptors, specifically, of one or both of the 5-HT_{1A} and 5-HT_{1B} receptors. They are useful in treating hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders), bipolar disorder (including in the depressed phase), attention-deficit/hyperactivity disorder (ADHD), and other disorders for which a 5-HT₁ agonist or antagonist is indicated.

European Patent Publication 434,561, published on Jun. 26, 1991, refers to 7-alkyl, alkoxy, and hydroxy substituted-1-(4-substituted-1-piperazinyl)-naphthalenes. The compounds are referred to as 5-HT₁ agonists and antagonists useful for the treatment of migraine, depression, anxiety, schizophrenia, stress and pain. European Patent Publication 343,050, published on Nov. 23, 1989, refers to 7-unsubstituted, halogenated, and methoxy substituted-1-(4-substituted-1-piper-azinyl)-naphthalenes as useful 5-HT_{1A} ligand therapeutics.

PCT publication WO 94/21619, published Sep. 29, 1994, refers to naphthalene derivatives as 5-HT₁ agonists and antagonists.

PCT publication WO 96/00720, published Jan. 11, 1996, now issued as U.S. Pat. No. 6,166,020 on Dec. 26, 2000 refers to naphthyl ethers as useful 5-HT₁ agonists and antagonists.

European Patent Publication 701,819, published Mar. 20, 1996, now issued as U.S. Pat. No. 5,597,826 on Jan. 28, 1997 refers to the use of 5-HT₁ agonists and antagonists in combination with a 5-HT re-uptake inhibitor.

Glennon et al., refers to 7-methoxy-1-(1-piperazinyl)-naphthalene as a useful 5-HT₁ ligand in their article “5-HT_{1B} Serotonin Receptors“, Drug Dev. Res., 22, 25-36 (1991).

Glennon's article “Serotonin Receptors: Clinical Implications”, Neuroscience and Behavioral Reviews, 14, 35-47 (1990), refers to the pharmacological effects associated with serotonin receptors including appetite suppression, thermoregulation, cardiovascular/hypotensive effects, sleep, psychosis, anxiety, depression, nausea, emesis, Alzheimer's disease, Parkinson's disease and Huntington's disease.

World Patent Application WO 95/31988, published Nov. 30, 1995, refers to the use of a 5-HT_{1B} antagonist in combination with a 5-HT_{1A} antagonist to treat CNS disorders such as depression, generalized anxiety, panic disorder, agoraphobia, social phobias, obsessive-compulsive disorder, post-traumatic stress disorder, memory disorders, anorexia nervosa and bulimia nervosa, Parkinson's disease, tardive dyskinesias, endocrine disorders such as hyperprolactinaemia, vasospasm (particularly in the cerebral vasculature) and hypertension, disorders of the gastrointestinal tract where changes in motility and secretion are involved, as well as sexual dysfunction.

G. Maura et al., J. Neurochem, 66 (1), 203-209 (1996), have stated that administration of agonists selective for 5-HT_{1A} receptors or for both 5-HT_{1A} and 5-HT_{1B} receptors might represent a great improvement in the treatment of human cerebellar ataxias, a multifaceted syndrome for which no established therapy is available.

European Patent Publication 666,261, published Aug. 9, 1995 refers to thiazine and thiomorpholine derivatives which are claimed to be useful for the treatment of cataracts.

All of the Foregoing World Patent Applications designate the United States. The foregoing patent and patent applications are incorporated by reference in their entirety.

### SUMMARY OF THE INVENTION

The present invention relates to compounds of the formula I 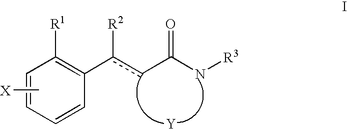
wherein R¹ is a group of the formula G¹, G², G³, G⁴, G⁵, G⁶, G⁷, G⁸ or G⁹ depicted below, 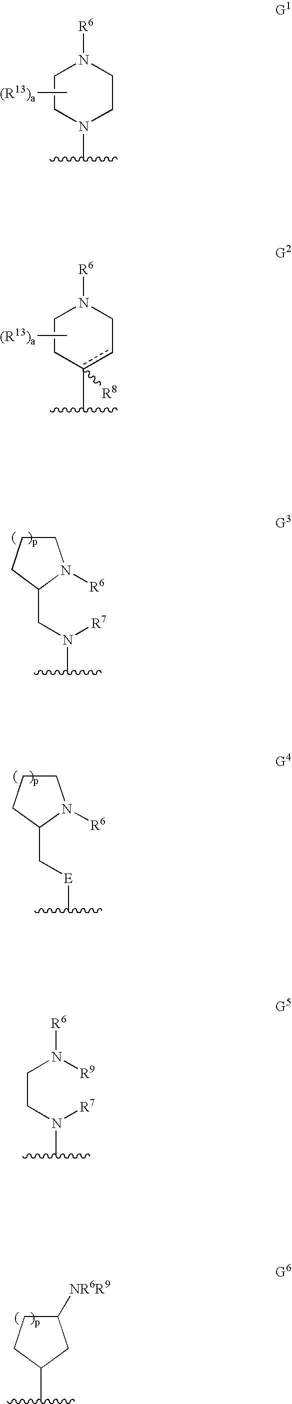 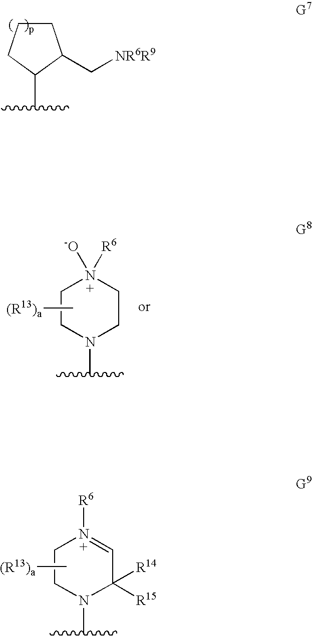
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —SOₜ(C₁-C₆)alkyl wherein t is zero one or two, —CO₂R¹⁰ or —CONR¹¹R¹²,
Y is an optionally substituted (C₁-C₄) heteroalkyl bridge that, together with the atoms to which it is attached, forms a six membered morpholin-3-on-2-yl ring; wherein the substituents on any of the carbon atoms capable of supporting an additional bond are fluoro, (C₁-C₆)alkyl, (C₁-C₆)alkoxy, trifluoromethyl or cyano;
R² is hydrogen, (C₁-C₄)alkyl, phenyl or naphthyl, wherein said phenyl or naphthyl may optionally be substituted with one or more substituents independently selected from chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;

R³ is —(CH₂)ₘB, wherein m is zero, one, two or three and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁶ is selected from the group consisting of hydrogen, (C₁-C₆)alkyl optionally substituted with (C₁-C₆)alkoxy or one to three fluorine atoms, or ((C₁-C₄)alkyl)aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, ((C₁-C₄)alkyl)aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that may contain from zero to four heteroatoms selected from nitrogen, sulfur and oxygen;
and p is one, two, or three;
each of R¹⁰, R¹¹ and R¹² is selected, independently, from the radicals set forth in the definition of R²; or R¹¹ and R¹², together with the nitrogen to which they are attached, form a 5 to 7 membered heteroalkyl ring that may contain from zero to four heteroatoms selected from nitrogen, sulfur and oxygen; and
the broken lines indicate optional double bonds, with the proviso that when the broken line in G² is a double bond that R⁸ is absent;
or a pharmaceutically acceptable salt thereof.

The following are more specific embodiments of groups G¹ and G².

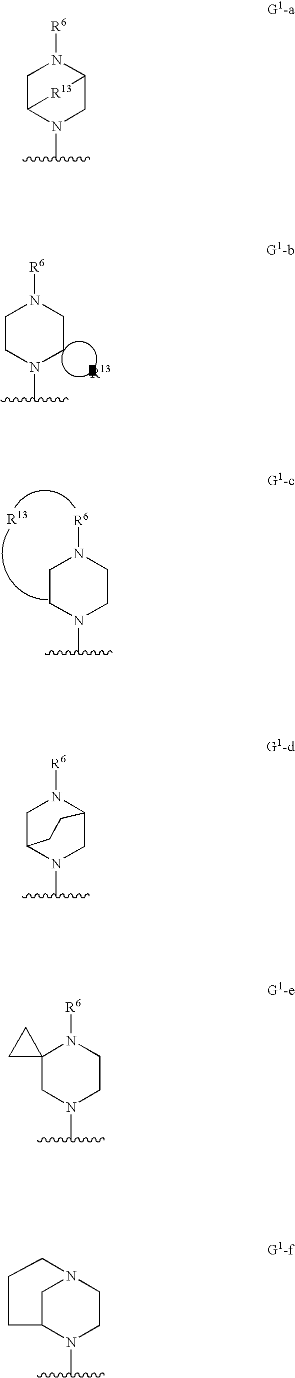

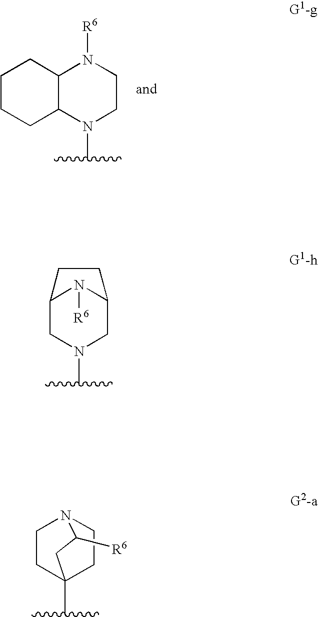

The present invention also relates to the pharmaceutically acceptable acid addition salts of compounds of the formula I. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate (i.e., 1,1′-methylene-bis-(2-hydroxy-3-naphthoate)) salts.

The invention also relates to base addition salts of formula I. The chemical bases that may be used as reagents to prepare pharmaceutically acceptable base salts of those compounds of formula I that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The compounds of this invention include all stereoisomers (e.g., cis (Z) and trans (E) isomers) and all optical isomers of compounds of the formula I (e.g., R and S enantiomers), as well as racemic, diastereomeric and other mixtures of such isomers. The compounds of this invention may contain olefin-like double bonds. When such bonds are present, the compounds of the invention exist as cis and trans configurations and as mixtures thereof.

Unless otherwise indicated, the alkyl and alkenyl groups referred to herein, as well as the alkyl moieties of other groups referred to herein (e.g., alkoxy), may be linear or branched, and they may also be cyclic (e.g., cyclopropyl, cyclobutyl, cyclopentyl, or cyclohexyl) or be linear or branched and contain cyclic moieties. Unless otherwise indicated, halogen includes fluorine, chlorine, bromine, and iodine.

Preferred compounds of the formula I include those wherein R¹ is 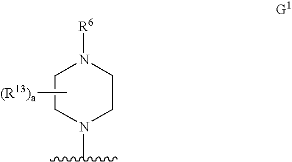
wherein R⁶ is methyl and R¹³ and R² are each hydrogen.

Other preferred compounds are those wherein R¹ is G⁶.

Preferred compounds of formula I include those wherein Y, together with the atoms to which it is attached, forms an optionally substituted morpholin-3-on-2-yl.

Preferred compounds of the formula I also include those wherein R³ is optionally substituted phenyl or —(CH₂)-optionally substituted phenyl.

Preferred compounds of the formula I are those of formula IA: 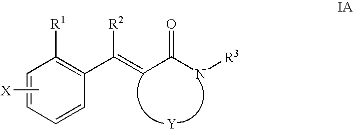
wherein X, Y, R¹, R² and R³ are as defined above, but where there is a double bond connecting the benzyl group to the lactam ring are those wherein the benzyl aromatic ring and the carbonyl group of the lactam ring are trans with respect to each other vis-a-vis the double bond.

Individual enantiomers of the compounds of formula I may have advantages, as compared with the racemic mixtures of these compounds, in the treatment of various disorders or conditions.

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed in the Schemes and/or in the Examples and Preparations below, by substituting a readily available isotopically labeled reagent for a non-isotopically labeled reagent.

Examples of specific preferred compounds of the formula I are the following:
2-[2-(4-Methylpiperazin-1-yl)-benzylidene]-4-(4-isopropylphenyl)-morpholin-3-one,
2-[2-(4-Methylpiperazin-1-yl)-benzylidene]-4-phenyl-morpholin-3-one,
2-[2-(4-Methylpiperazin-1-yl)-benzylidene]-4-(4-tert-butylphenyl)-morpholin-3-one,
2-[2-(3,4,5-Trimethylpiperazin-1-yl)-benzylidene]-4-(4-tert-butylphenyl)-morpholin-3-one,
4-[4-(1-Hydroxy-1-methylethyl)-phenyl]-2-[2-(4-methylpiperazin-1-yl)-benzylidene]-morpholin-3-one,
4-(4-tert-Butyl-phenyl)-2-[4-fluoro-2-(4-methylpiperazin-1-yl)-benzylidene]-morpholin-3-one,
4-(4-tert-Butyl-phenyl)-2-[6-chloro-2-(4-methylpiperazin-1-yl)-benzylidene]-morpholin-3-one,
4-(4-tert-Butyl-phenyl)-2-[4-chloro-2-(4-methylpiperazin-1-yl)-benzylidene]-morpholin-3-one,
4-(4-tert-Butyl-phenyl)-2-[6-fluoro-2-(4-methylpiperazin-1-yl)-benzylidene]-morpholin-3-one,
4-(4-tert-Butyl-phenyl)-2-[3-fluoro-2-(4-methylpiperazin-1-yl)-benzylidene]-morpholin-3-one,
4-(4-tert-Butyl-phenyl)-2-[2-(4-methylpiperazin-1-yl)-6-trifluoromethyl-benzylidene]-morpholin-3-one,
4-(4-tert-Butyl-phenyl)-2-[2-(4-methylpiperazin-1-yl)-4-trifluoromethyl-benzylidene]-morpholin-3-one,
4-(4-tert-Butyl-phenyl)-2-[5-methyl-2-(4-methylpiperazin-1-yl)-benzylidene]-morpholin-3-one,
4-(4-tert-Butyl-phenyl)-2-[2-(3-(R)-dimethylamino-pyrrolidin-1-yl)-benzylidene]-morpholin-3-one,
4-(4-tert-Butyl-benzyl)-2-[2-(4-methylpiperazin-1-yl)-benzylidene]-morpholin-3-one,
4-(4-Chlorobenzyl)-5-methyl-2-[2-(4-methylpiperazin-1-yl)-benzylidene]-morpholin-3-one,
(±)-4-(4-tert-Butyl-phenyl)-2-[2-(4-methylpiperazin-1-yl)-benzyl]-morpholin-3-one,
(+)-4-(4-tert-Butyl-phenyl)-2-[2-(4-methylpiperazin-1-yl)-benzyl]-morpholin-3-one,
(−)-4-(4-tert-Butyl-phenyl)-2-[2-(4-methylpiperazin-1-yl)-benzyl]-morpholin-3-one,
(±)-4-(4-Isopropyl-phenyl)-2-[2-(4-methylpiperazin-1-yl)-benzyl]-morpholin-3-one,
(−)-4-(4-Isopropyl-phenyl)-2-[2-(4-methylpiperazin-1-yl)-benzyl]-morpholin-3-one,
(+)-4-(4-Isopropyl-phenyl)-2-[2-(4-methylpiperazin-1-yl)-benzyl]-morpholin-3-one,
(±)-4-(4-tert-Butyl-phenyl)-2-[2-chloro-6-(4-methylpiperazin-1-yl)-benzyl]-morpholin-3-one,
(+)-4-(4-tert-Butyl-phenyl)-2-[2-chloro-6-(4-methylpiperazin-1-yl)-benzyl]-morpholin-3-one,
(−)-4-(4-tert-Butyl-phenyl)-2-[2-chloro-6-(4-methylpiperazin-1-yl)-benzyl]-morpholin-3-one,
(±)-4-(4-tert-Butyl-phenyl)-2-[4-chloro-2-(4-methylpiperazin-1-yl)-benzyl]-morpholin-3-one,
(+)-4-(4-tert-Butyl-phenyl)-2-[4-chloro-2-(4-methylpiperazin-1-yl)-benzyl]-morpholin-3-one,
(−)-4-(4-tert-Butyl-phenyl)-2-[4-chloro-2-(4-methylpiperazin-1-yl)-benzyl]-morpholin-3-one,
(±)-4-(4-tert-Butyl-phenyl)-2-[2-fluoro-6-(4-methylpiperazin-1-yl)-benzyl]-morpholin-3-one,
(+)-4-(4-tert-Butyl-phenyl)-2-[2-fluoro-6-(4-methylpiperazin-1-yl)-benzyl]-morpholin-3-one,
(−)-4-(4-tert-Butyl-phenyl)-2-[2-fluoro-6-(4-methylpiperazin-1-yl)-benzyl]-morpholin-3-one,
(±)-4-(4-tert-Butyl-phenyl)-2-[4-fluoro-2-(4-methylpiperazin-1-yl)-benzyl]-morpholin-3-one,
(+)-4-(4-tert-Butyl-phenyl)-2-[4-fluoro-2-(4-methylpiperazin-1-yl)-benzyl]-morpholin-3-one,
(−)-4-(4-tert-Butyl-phenyl)-2-[4-fluoro-2-(4-methylpiperazin-1-yl)-benzyl]-morpholin-3-one,
(±)-4-(4-tert-Butyl-phenyl)-2-[3-fluoro-2-(4-methylpiperazin-1-yl)-benzyl]-morpholin-3-one,
(+)-4-(4-tert-Butyl-phenyl)-2-[3-fluoro-2-(4-methylpiperazin-1-yl)-benzyl]-morpholin-3-one,
(−)-4-(4-tert-Butyl-phenyl)-2-[3-fluoro-2-(4-methylpiperazin-1-yl)-benzyl]-morpholin-3-one,
(±)-4-(4-tert-Butyl-phenyl)-2-[5-methyl-2-(4-methylpiperazin-1-yl)-benzyl]-morpholin-3-one,
(+)-4-(4-tert-Butyl-phenyl)-2-[5-methyl-2-(4-methylpiperazin-1-yl)-benzyl]-morpholin-3-one,
(−)-4-(4-tert-Butyl-phenyl)-2-[5-methyl-2-(4-methylpiperazin-1-yl)-benzyl]-morpholin-3-one,
(±)-4-(4-tert-Butyl-phenyl)-2-[2-(4-methylpiperazin-1-yl)-5-trifluoromethyl-benzyl]-morpholin-3-one,
(+)-4-(4-tert-Butyl-phenyl)-2-[2-(4-methylpiperazin-1-yl)-5-trifluoromethyl-benzyl]-morpholin-3-one,
(−)-4-(4-tert-Butyl-phenyl)-2-[2-(4-methylpiperazin-1-yl)-5-trifluoromethyl-benzyl]-morpholin-3-one,
(±)-4-Biphenyl-4-yl-2-[2-(4-methylpiperazin-1-yl)-benzyl]-morpholin-3-one,
(±)-4-(4-tert-Butyl-phenyl)-2-[2-(3,4,5-trimethylpiperazin-1-yl)-benzyl]-morpholin-3-one,
(+)-4-(4-tert-Butyl-phenyl)-2-[2-(3,4,5-trimethylpiperazin-1-yl)-benzyl]-morpholin-3-one,
(−)-4-(4-tert-Butyl-phenyl)-2-[2-(3,4,5-trimethylpiperazin-1-yl)-benzyl]-morpholin-3-one,
(±)-4-(4-tert-Butyl-benzyl)-2-[2-(4-methyl-piperazin-1-yl)-benzyl]-morpholin-3-one,
2-[2-(4-Methylpiperazin-1-yl)-benzyl]-4-(4-trifluoromethyl-phenyl)-morpholin-3-one and the pharmaceutically acceptable salts of such compounds.

Other compounds of formula I include the following:
4-(4-tert-Butyl-phenyl)-2-[2-(3,5-dimethylpiperazin-1-yl)-benzyl]-morpholin-3-one,
4-(4-tert-Butyl-phenyl)-2-[2-(3,3,5,5-tetramethylpiperazin-1-yl)-benzyl]-morpholin-3-one,
4-(4-tert-Butyl-phenyl)-2-{2-[methyl-( 1-methylpyrrolidin-3-yl)-amino]-benzyl}-morpholin-3-one,
4-(4-tert-Butyl-phenyl)-5-methyl-2-[2-(4-methylpiperazin-1-yl)-benzyl]-morpholin-3-one,
4-(4-tert-Butyl-phenyl)-2-[4-methoxy-2-(4-methylpiperazin-1-yl)-benzyl]-morpholin-3-one,
4-(4-tert-Butyl-phenyl)-2-[4-hydroxy-2-(4-methylpiperazin-1-yl)-benzyl]-morpholin-3-one,
4-(4-tert-Butyl-phenyl)-2-[4-methanesulfonyl-2-(4-methylpiperazin-1-yl)-benzyl]-morpholin-3-one,
2-[4-Bromo-2-(4-methylpiperazin-1-yl)-benzyl]-4-(4-tert-butyl-phenyl)-morpholin-3-one,
4-(4-tert-Butyl-phenyl)-2-[4-iodo-2-(4-methylpiperazin-1-yl)-benzyl]-morpholin-3-one,
4-(4-Cyclopropyl-phenyl)-2-[2-(4-methylpiperazin-1-yl)-benzyl]-morpholin-3-one,
4-[4-(3-Methyl-cyclobutyl)-phenyl]-2-[2-(4-methylpiperazin-1-yl)-benzyl]-morpholin-3-one,
4-(4-Cyclopentyl-phenyl)-2-[2-(4-methylpiperazin-1-yl)-benzyl]-morpholin-3-one,
4-(4-Cyclopentyl-phenyl)-2-[2-(4-ethylpiperazin-1-yl)-benzyl]-morpholin-3-one,
4-[4-(4,4-Dimethyl-cyclohexyl)-phenyl]-2-[2-(4-methylpiperazin-1-yl)-benzyl]-morpholin-3-one,
4-[4-(1-Methyl-cyclohexyl)-phenyl]-2-[2-(4-methylpiperazin-1-yl)-benzyl]-morpholin-3-one,
4-(4-Cyclohexyl-3-methyl-phenyl)-2-[2-(4-methylpiperazin-1-yl)-benzyl]-morpholin-3-one,
4-(4-Cyclohexyl-3-methyl-phenyl)-2-[2-(4-methylpiperazin-1-yl)-benzyl]-morpholin-3-one,
4-{4-[2-(4-Fluorophenyl)-cyclopropyl]-phenyl}-2-[2-(4-methylpiperazin-1-yl)-benzyl]-morpholin-3-one,
4-[4-(4-Fluorobenzyl)-phenyl]-2-[2-(4-methylpiperazin-1-yl)-benzyl]-morpholin-3-one,
4-(6-Isopropyl-naphthalen-2-yl)-2-[2-(4-methylpiperazin-1-yl)-benzyl]-morpholin-3-one,
4-(6-tert-Butyl-naphthalen-2-yl)-2-[2-(4-methylpiperazin-1-yl)-benzyl]-morpholin-3-one,
4-[1-(4-tert-Butyl-phenyl)-ethyl]-2-[2-(4-methylpiperazin-1-yl)-benzyl]-morpholin-3-one,
4-(5-tert-Butyl-thiophen-2-ylmethyl)-2-[2-(4-methylpiperazin-1-yl)-benzyl]-morpholin-3-one,
4-(5-tert-Butyl-1-oxo-1H-thiophen-2-ylmethyl)-2-[2-(4-methylpiperazin-1-yl)-benzyl]-morpholin-3-one,
4-(5-tert-Butyl-1,1-dioxo-1H-thiophen-2-ylmethyl)-2-[2-(4-methylpiperazin-1-yl)-benzyl]-morpholin-3-one,
4-(4-tert-Butyl-benzyl)-2-[4-chloro-2-(3-dimethylamino-pyrrolidin-1-yl)-benzyl]-morpholin-3-one,
2-[4-Chloro-2-(3-dimethylamino-pyrrolidin-1-yl)-benzyl]-4-(4-isopropyl-phenyl)-morpholin-3-one,
2-[2-(3-Dimethylamino-azetidin-1-yl)-4-fluorobenzyl]-4-(4-isopropyl-phenyl)-morpholin-3-one,
4-(4-tert-Butyl-phenyl)-2-[2-(3-dimethylamino-pyrrolidin-1-yl)-4-fluoro-5-isopropyl-benzyl]-morpholin-3-one and
2-[2-(3-Dimethylamino-pyrrolidin-1-yl)-benzyl]-4-[4-(1-methyl-cyclopentyl)-phenyl]-morpholin-3-one.

The present invention also relates to intermediates of the formula V: 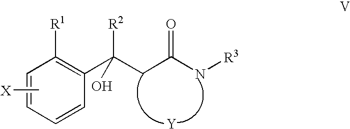
wherein R¹, R², R³, X, and Y are as defined above. Also included are all optical isomers of compounds of the formula V (e.g., R and S enantiomers), as well as racemic, diastereomeric and other mixtures of such isomers. The compounds of this invention may contain olefin-like double bonds. When such bonds are present, the compounds of the invention exist as cis and trans configurations and as mixtures thereof.

Examples of specific preferred compounds of formula V are the following:
4-(4-tert-Butyl-phenyl)-2-{hydroxy-[2-(4-methylpiperazin-1-yl)-phenyl]-methyl}-morpholin-3-one,
4-(4-tert-Butyl-phenyl)-2-{[4-fluoro-2-(4-methylpiperazin-1-yl)-phenyl]-hydroxymethyl}-morpholin-3-one,
4-(4-tert-Butyl-phenyl)-2-{1-[4-fluoro-2-(4-methylpiperazin-1-yl)-phenyl]-1-hydroxy-ethyl}-morpholin-3-one,
4-[4-(1,1-Dimethylpropyl)-phenyl]-2-{1-hydroxy-1-[2-(4-methylpiperazin-1-ethyl}-morpholin-3-one,
4-(4-tert-Butyl-phenyl)-2-{[6-fluoro-2-(4-methylpiperazin-1-yl)-phenyl]-hydroxymethyl}-morpholin-3-one,
4-(4-tert-Butyl-phenyl)-2-{[4-chloro-2-(4-methylpiperazin-1-yl)-phenyl]-hydroxymethyl}-morpholin-3-one,
4-(4-Isopropyl-phenyl)-2-{[4-chloro-2-(4-methylpiperazin-1-yl)-phenyl]-hydroxy-methyl}-morpholin-3-one and
4-(4-Isopropyl-phenyl)-2-[2-(4-methylpiperazin-1-yl)-phenyl]-hydroxymethyl-morpholin-3-one.

Other preferred compounds of the invention are those of formula IB 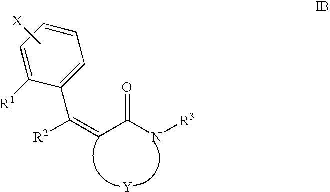
wherein, X, Y, R¹, R², and R³ are as defined above. These compounds of formula IB are isomers of the compounds of formula IA wherein there is a double bond connecting the benzyl group to the lactam ring and wherein the benzyl aromatic ring and the carbonyl group of the lactam ring are cis with respect to each other vis-a-vis the double bond. The present invention accordingly encompasses those groups of compounds and species as set forth above with the geometric structure of formula IB.

The present invention also relates to a pharmaceutical composition for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders), bipolar disorder (including in the depressed phase), and attention-deficit/hyperactivity disorder (ADHD), in a mammal, preferably a human, comprising (a) an amount of a compound of the formula I or a pharmaceutically acceptable salt thereof (b) and a pharmaceutically acceptable carrier effective in treating such disorder or condition.

The present invention also relates to a pharmaceutical composition for treating a disorder or condition that can be treated by enhancing serotonergic neurotransmission in a mammal, preferably a human, comprising an amount of a compound of the formula I, or a pharmaceutically acceptable salt thereof, effective in treating such disorder or condition and a pharmaceutically acceptable carrier. Examples of such disorders and conditions are those enumerated in the preceding paragraph.

The present invention also relates to a pharmaceutical composition for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, in a mammal, preferably a human, comprising (a) an amount of a compound of the formula I or a pharmaceutically acceptable salt thereof and (b) a pharmaceutically acceptable carrier effective in treating such disorder or condition.

The present invention also relates to a method for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders), bipolar disorder (including in the depressed phase), and attention-deficit/hyperactivity disorder (ADHD), in a mammal, preferably a human, comprising administering to a mammal in need of such treatment an amount of a compound of the formula I, or a pharmaceutically acceptable salt thereof, that is effective in treating such disorder or condition.

The present invention also relates to a method for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, in a mammal, preferably a human, comprising administering to a mammal in need of such treatment an amount of a compound of the formula I, or a pharmaceutically acceptable salt thereof, that is effective in treating such disorder or condition.

The present invention also relates to a method for treating a disorder or condition that can be treated by enhancing serotonergic neurotransmission in a mammal, preferably a human, comprising administering to a mammal in need of such treatment an amount of a compound of the formula I, or a pharmaceutically acceptable salt thereof, that is effective in treating such disorder or condition.

The present invention also relates to a pharmaceutical composition for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders), bipolar disorder (including in the depressed phase), and attention-deficit/hyperactivity disorder (ADHD), in a mammal, preferably a human, comprising a serotonin receptor antagonizing or agonizing effective amount of a compound of the formula I, or a pharmaceutically acceptable salt thereof, and a pharmaceutically acceptable carrier.

The present invention also relates to a pharmaceutical composition for treating a disorder or condition that can be treated by enhancing serotonergic neurotransmission in a mammal, preferably a human, comprising a serotonin receptor antagonizing or agonizing effective amount of a compound of the formula I, or a pharmaceutically acceptable salt thereof, and a pharmaceutically acceptable carrier.

The present invention also relates to a pharmaceutical composition for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, in a mammal, preferably a human, comprising a serotonin receptor antagonizing or agonizing effective amount of a compound of the formula I, or a pharmaceutically acceptable salt thereof, and a pharmaceutically acceptable carrier.

The present invention also relates to a method for treating a disorder or condition selected from hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders), bipolar disorder (including in the depressed phase), and attention-deficit/hyperactivity disorder (ADHD), in a mammal, preferably a human, comprising administering to a mammal requiring such treatment a serotonin receptor antagonizing or agonizing effective amount of a compound of the formula I or a pharmaceutically acceptable salt thereof.

The present invention also relates to a method for treating a disorder or condition that can be treated by enhancing serotonergic neurotransmission in a mammal, preferably a human, comprising administering to a mammal requiring such treatment a serotonin receptor antagonizing or agonizing effective amount of a compound of the formula I or a pharmaceutically acceptable salt thereof.

The present invention also relates to a method for treating a disorder or condition selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, preferably a human, comprising administering to a mammal requiring such treatment a serotonin receptor antagonizing or agonizing amount of a compound of the formula I, or a pharmaceutically acceptable salt thereof.

The compounds of the present invention are also useful in the treatment of patients afflicted with two or more of the above disorders. It is not uncommon for certain of the above listed disorders, which can be treated using the novel compounds of the invention, to exist in patients afflicted with one or more other such disorders. For example, depression is often co-morbid with anxiety and both may be treated using the compounds or pharmaceutical compositions of the present invention.

A further particular advantage of the serotonin 1 (5-HT₁) receptor agonist/antagonist compounds of the present invention is that they exhibit pharmacological and therapeutic activity without the delayed onset of action usually associated with selective serotonin reuptake inhibitors.

The present invention further relates to a pharmaceutical composition for treating a condition or disorder that can be treated by enhancing serotonergic neurotransmission in a mammal, preferably a human, comprising:
a) a compound of the formula I or a pharmaceutically acceptable salt thereof; and
b) a 5-HT re-uptake inhibitor, or a pharmaceutically acceptable salt thereof; and
c) a pharmaceutically acceptable carrier;
wherein the amount of the active agents “a” and “b” above are present in amounts that render the composition effective in treating respectively such a disorder or condition.

The present invention also relates to a method for treating a disorder or condition that can be treated by enhancing serotonergic neurotransmission in a mammal, preferably a human, comprising administering to a mammal requiring such treatment:
a) a compound of the formula I, defined above, or a pharmaceutically acceptable salt thereof; and
b) a 5-HT re-uptake inhibitor, or a pharmaceutically acceptable salt thereof;
wherein the amounts of the active agents “a” and “b” above are present in amounts that render the combination of the two agents effective in treating such a disorder or condition.

The present invention also relates to a method for treating a disorder or condition that can be treated by enhancing serotonergic neurotransmission in a mammal, preferably a human, comprising administering to said mammal requiring such treatment:
a) a 5-HT_{1A} antagonist or a pharmaceutically acceptable salt thereof; and
b) a 5-HT_{1B} antagonist of formula I or a pharmaceutically acceptable salt thereof;
wherein the amounts of each active compound “a” and “b” are present in amounts that render the combination of the two agents effective in treating respectively such a disorder or condition.

The present invention also relates to a pharmaceutical composition for treating a disorder or condition that can be treated by enhancing serotonergic neurotransmission in a mammal, preferably a human, comprising:
a) a 5-HT_{1A} antagonist or a pharmaceutically acceptable salt thereof; and
b) a 5-HT_{1B} antagonist of formula I or a pharmaceutically acceptable salt thereof; and
c) a pharmaceutically acceptable carrier;
wherein the amounts of each active compound “a” and “b” above are present in amounts that render the composition effective in treating respectively such a disorder or condition.

“Treating” refers to, and includes, reversing, alleviating, inhibiting the progress of, or preventing, a disease, disorder or condition, or one or more symptoms thereof; and, “treatment” and “therapeutically” refer to the act of treating, as defined above.

“Enhanced serotonergic neurotransmission,” as used herein, refers to increasing or improving the neuronal process whereby serotonin is released by a pre-synaptic cell upon excitation and crosses the synapse to stimulate or inhibit the post-synaptic cell.

“Chemical dependency,” as used herein, means an abnormal craving or desire for, or an addiction to, a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, heroin, phenobarbital, and benzodiazepines (e.g., Valium (trademark)). “Treating a chemical dependency,” as used herein, means reducing or alleviating such dependency.

The preferred 5-HT reuptake inhibitor sertraline, (1S-cis)-4-(3,4-dichlorophenyl)-1,2,3,4-tetrahydro-N-methyl-1-naphthalenamine, as used herein has the following structural formula 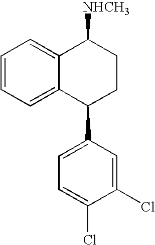
and is ordinarily used in the form of its hydrochloride salt. The synthesis of sertraline is described in U.S. Pat. No. 4,536,518, assigned to Pfizer Inc. Sertraline hydrochloride is useful as an antidepressant and anorectic agent, and is also useful in the treatment of depression, chemical dependencies, anxiety, obsessive-compulsive disorders, phobias, panic disorder, post-traumatic stress disorder, and premature ejaculation. The foregoing patent is incorporated by reference in its entirety.

### DETAILED DESCRIPTION OF THE INVENTION

Compounds of the formula I may be prepared according to the following reaction schemes and discussion. Unless otherwise indicated, R¹ through R³, R⁶ through R¹⁵, G¹ through G⁹, X, B, E, Y, Z, g, j, k, m, n, p, q, r and t and structural formula I in the reaction schemes and discussion that follow are as defined above.

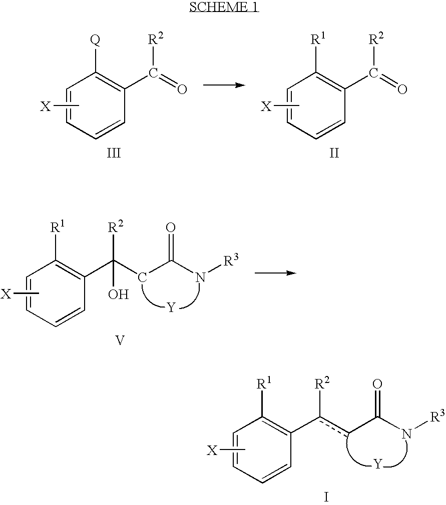

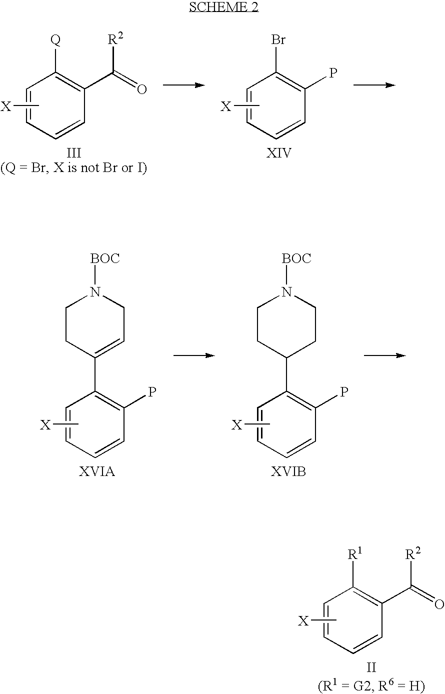

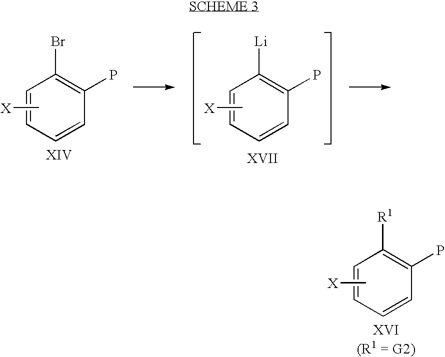

Scheme 1 illustrates a method of synthesizing compounds of the formula I wherein the dashed line represents a double carbon-carbon bond and R¹ is a group of the formula G¹, G³, G⁴, G⁵, G⁶ or G⁷. Referring to Scheme 1, a compound of the formula III, wherein Q is a suitable leaving group (e.g., chloro, fluoro, bromo, mesylate, tosylate, etc.), is reacted with a compound of the formula R¹H, wherein H refers to a hydrogen atom on group E or on nitrogen atoms from G¹, G³, G⁵, G⁶ or G⁷ and R¹ is a group of the formula G¹, G³, G⁴, G⁵, G⁶ or G⁷ in the presence of a base, to form the corresponding compound of formula II. This reaction is generally carried out at a temperature from about 0° C. to about 140° C., preferably at about the reflux temperature, in a polar solvent such as dimethyl sulfoxide (DMSO), N,N-dimethylformamide (DMF), N,N-dimethylacetamide (DMA) or N-methyl-2-pyrrolidinone (NMP), preferably DMF. Suitable bases include anhydrous sodium carbonate (Na₂CO₃), potassium carbonate (K₂CO₃), sodium hydroxide (NaOH) and potassium hydroxide (KOH), as well as amines such as pyrrolidine, triethylamine and pyridine. Anhydrous potassium carbonate is preferred.

Compounds of formula II can be converted into compounds of the formula I, wherein R³ is other than hydrogen, by subjecting them to an aldol condensation or Wittig reaction. For example, in the case of an aldol condensation, a compound of the formula II can be reacted with a compound of the formula IV: 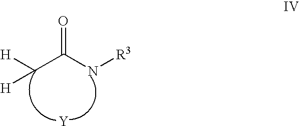
in the presence of a base, to form an aldol intermediate of the formula V 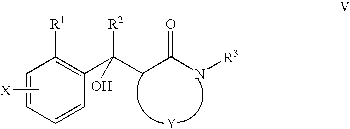
which may be isolated or converted directly in the same reaction step to a compound of the formula I by the loss of water. The degree of completion for the conversion of compounds of the formula II to the aldol product of formula I may be assessed using one or more analytical techniques, such as thin layer chromatography (tlc) or mass spectrometry. In some instances it may be possible or desirable to isolate the intermediate of formula V. In such case, the compound of formula V may be converted into the compound of formula I by the elimination of water using techniques which are familiar to those skilled in the art, for example, by heating to the reflux temperature a solution of the compound of formula V in a solvent such as benzene, toluene or xylene, in the presence of a catalytic amount of benzene- or p-toluene-sulfonic acid with provision for the removal of the water generated. Such water removal techniques may involve the use of molecular sieves or a Dean-Stark trap to isolate the water created as an azeotrope with the solvent.

The aldol reaction is typically carried out in a polar solvent such as DMSO, DMF, tetrahydrofuran (THF), methanol or ethanol, at a temperature from about −78° C. to about 80° C. Preferably, this reaction is carried out in THF at about 25° C. Suitable bases for use in the aldol formation step include potassium carbonate (K₂CO₃), sodium carbonate (Na₂CO₃), sodium hydride (NaH), sodium methoxide, sodium ethoxide, potassium-tert-butoxide, lithium diisopropylamide, pyrrolidine and piperidine. Sodium hydride is preferred. Aldol condensations are described in “Modern Synthetic Reactions,” Herbert O. House, 2d. Edition, W. A. Benjamin, Menlo Park, Calif., 629-682 (1972) and Tetrahedron, 38 (20), 3059 (1982).

Compounds of the formula I, wherein R³ is other than hydrogen, can also be prepared from compounds of formula II by reaction with a compound of the formula IV, wherein R³ is hydrogen or —(C═O)R¹³, wherein R¹³ is (C₁-C₆)alkyl or trifluoromethyl, followed by removal of the —C(═O)R¹³ group, if present, and reaction with a compound of the formula R³-L′ wherein L′ is a leaving group and is defined as Q is defined as above. These reactions can be carried out in a solvent such as di-(alkyl)ether, THF, DMF, DMA or DMSO, preferably DMF, in the presence of a base such as potassium carbonate, sodium carbonate, sodium hydride, potassium hydride, sodium hydroxide or potassium hydroxide, preferably sodium hydride. Reaction temperatures can range from about 0° C. to about 150° C., preferably from about 25° C. to about the reflux temperature of the solvent.

Alternatively, the compound of formula IV can be converted into a compound of the formula I by means of a Wittig olefination, as described in Helvetica Chimica Acta, 46, 1580 (1963), and depicted below.

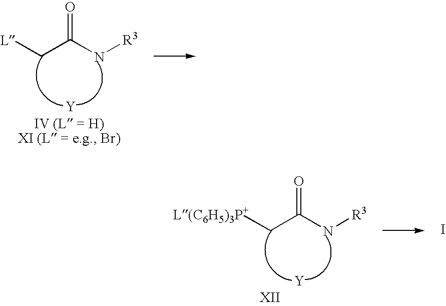

Thus, the compound of formula IV can be converted into the corresponding bromide of formula XI using standard bromination conditions, followed by treatment with triphenylphosphine in anhydrous THF to form the intermediate of formula XII. The compound of formula XII can then be treated with a base (e.g., aqueous Na₂CO₃) to generate the corresponding phosphonium ylide, which can then be reacted with the appropriate intermediate of formula II to produce compounds of general formula I. This transformation is described in A. Maercker, Organic Reactions, 14, 270 (1965).

Compounds of the formula I wherein the dashed line represents a single carbon-carbon bond may be prepared by hydrogenating the corresponding compounds wherein the dashed line represents a double carbon-carbon bond, using standard techniques that are well known to those skilled in the art. For example, reduction of the double bond may be effected with hydrogen gas (H₂), using catalysts such as palladium on carbon (Pd/C), palladium on barium sulfate (Pd/BaSO₄), platinum on carbon (Pt/C), or tris(triphenylphosphine) rhodium chloride (Wilkinson's catalyst), in an appropriate solvent such as methanol, ethanol, THF, dioxane or ethyl acetate, at a pressure from about 1 to about 5 atmospheres and a temperature from about 10° C. to about 60° C., as described in Catalytic Hydrogenation in Organic Synthesis, Paul Rylander, Academic Press Inc., San Diego, 31-63 (1979). The following conditions are preferred: Pd on carbon, methanol at 25° C. and 50 psi of hydrogen gas pressure. This method also provides for introduction of hydrogen isotopes (i.e., deuterium, tritium) by replacing ¹H₂ with ²H₂ or ³H₂ in the above procedure.

An alternative procedure employing the use of reagents such as ammonium formate and Pd/C in methanol at the reflux temperature under an inert atmosphere (e.g., nitrogen or argon gas) is also effective in reducing the carbon-carbon double bond of compounds of the formula I. Another alternative method involves selective reduction of the carbon-carbon double bond. This can be accomplished using samarium and iodine or samarium iodide (SmI₂) in methanol or ethanol at about room temperature, as described by R. Yanada et. al., Synlett., 443-4 (1995).

The starting materials of the formulas III and IV are either commercially available or known in the art. For example, compounds of formula III in which R² is hydrogen are readily available from commercial sources or may be prepared using procedures disclosed in the chemical literature. Compounds of the formula III may also be prepared from the corresponding carboxylic acids or esters, (i.e., formula III) wherein R²═OH or O-alkyl), which are commercially available. These acids or esters can be reduced to the corresponding alcohols of formula XIII, depicted below, wherein Q is defined as for formula III, using one or more of a variety of reducing agents and conditions, depending upon the nature of the substituents Q and X.

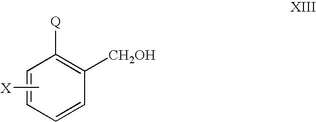

Such reducing agents include sodium borohydride (NaBH₄), sodium cyanoborohydride (NaCNBH₃), lithium aluminum hydride (LiAlH₄) and borane, in THF (BH₃.THF) in solvents such as methanol, ethanol, THF, diethyl ether and dioxane. Oxidation of the alcohol of formula XIII to the corresponding aldehyde of formula II may be accomplished using a selective oxidizing agent such as Jones reagent (hydrogen chromate (H₂CrO₄)), pyridinium chlorochromate (PCC) or manganese dioxide (MnO₂). References for such conversions are readily available (e.g., K. B. Wiberg, Oxidation in Organic Chemistry, Part A, Academic Press Inc, N.Y., 69-72 (1965)).

Compounds of the formula IV, wherein R³ is hydrogen (compounds of the formula IVA), may be alkylated to form the corresponding compounds wherein R³ is not hydrogen using standard techniques available to those skilled in the art, e.g., by (a) generation of the anion of the desired compound of formula IVA using a strong base/polar solvent system such as NaH/THF, NaH/DMF or n-butyllithium/THF (n-BuLi/THF), at a temperature from about −30° C. to about the reflux temperature of the solvent, for a period of about 5 minutes to about 24 hours, and (b) treatment of the anion with an alkylating agent of the formula R³L′ wherein L′ is a leaving group such as chloro, bromo, iodo or mesylate. This process is depicted below.

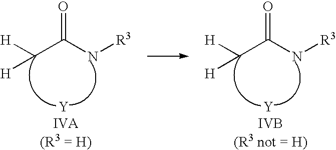

The foregoing conversion of compounds of the formula IVA to those of the formula IVB may also be achieved using phase transfer catalysis conditions as described by Takahata et al., Heterocycles, 1979, 12(11), pp. 1449-1451.

Compounds of the formula IVB wherein R³ is aryl or heteroaryl can be prepared from compounds of the formula IVA by reaction with an aryl or heteroaryl reagent of the formula R³L′, wherein L′ is a leaving group such as chloro, bromo or iodo, in the presence of a catalyst such as copper (0) or copper (I) (such as copper, copper-bronze, or copper bromide) and a base, such as sodium hydride, potassium carbonate, or sodium carbonate. The reaction may be run neat or with a polar solvent such as dimethyl formamide, or dimethyl sulfoxide. This reaction, referred to as an Ullmann condensation, is described by Yamamoto & Kurata, Chem. and Industry, 737-738 (1981).

Alternatively, compounds of the formula IVB wherein R³ is aryl or heteroaryl can be prepared from compounds of the formula IVD, which are commercially available or prepared according to the method of S. L. Buchwald et al in the Journal of Organic Chemistry, 2000, 65(4), pp. 1144-1157 starting with compounds of formula IVC (e.g., morpholine) and a suitable aryl or heteroaryl bromide (R³L′). This intermediate of formula IVD can then be oxidized to the intermediates of formula IVB using a suitable oxidizing agent, such as potassium permanganate, in the presence of a quaternary ammonium compound, such as benzyltriethylammonium chloride, in a reaction inert solvent such as methylene chloride, chloroform or toluene, according to the procedure described by J. H. Markgraf and C. A. Stickney in the Journal of Heterocyclic Chemistry, 2000, 37(11), pp. 109-110.

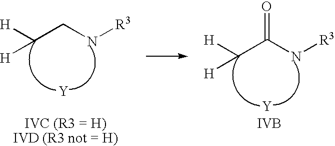

The compounds of formula R¹H used in the preparation of intermediates of the formula II are readily available or may be prepared using standard methods of organic synthesis known to those skilled in the art and adapted from procedures disclosed in the chemical literature. For example, the preparation of compounds of the formula R¹H, wherein R¹ is G¹, may be accomplished using the following reaction sequence, beginning with commercially available N-tert-butoxycarbonyl piperazine (VI):

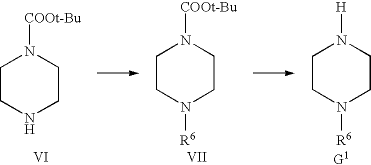

Alkylation of the compound of formula VI with a compound of the formula R⁶L′ wherein L′ is a leaving group, and is defined as Q is defined above and R⁶ is (C₁-C₆)alkyl, aryl-(C₁-C₄)alkyl wherein the aryl moiety is phenyl or naphthyl, or heteroaryl-(CH₂)_{q}—, wherein q is zero, one, two, three or four, and the heteroaryl moiety is selected from pyridyl, pyrimidyl, benzoxazolyl, benzothiazolyl, benzisoxazolyl, and benzisothiazolyl, in the presence of an acid scavenger (e.g., sodium bicarbonate (NaHCO₃), potassium bicarbonate (KHCO₃), sodium carbonate (Na₂CO₃) or potassium carbonate (K₂CO₃)), in a polar solvent such as acetone at a temperature of about 10° C. to about the reflux temperature of the solvent, will yield the intermediate of formula VII. Removal of the tert-butoxycarbonyl group can be accomplished using acidic conditions, e.g., HBr in acetic acid or trifluoroacetic acid until the reaction is judged to be complete.

Compounds of the formula II, wherein R¹ is tetrahydropyridine or piperidine (i.e. compounds of the formula G²) and R² is hydrogen, can be prepared from the 2-bromobenzaldehyde of formula III, many of which are commercially available, as depicted in Scheme 2. Referring to Scheme 2, the compound of formula III is first converted into a protected aldehyde of the formula XIV, wherein P represents the entire protected aldehyde or ketone moiety, using methods well known in the art. For example, the 1,3-dioxolane derivative of the aldehyde may be prepared according to the method described by J. E. Cole et al., J. Chem. Soc., 244 (1962), by refluxing a solution of the aldehyde of formula III and 1,3-propanediol in anhydrous benzene with a catalytic amount of p-toluenesulfonic acid. When R² of formula III is not hydrogen, the ketone can be protected using an appropriate protecting group. Appropriate protecting groups can be chosen from many such groups based on the presence and nature of the substituent X. Examples of suitable protecting groups may be found in T. W. Greene and P. Wuts, Protecting Groups in Organic Synthesis, John Wiley & Sons, 2nd Edition, New York, 1991. The most preferred protecting groups are those that are resistant to catalytic hydrogenation (e.g., 1,3-dioxolane), which would therefore allow for the subsequent reduction, if required, of the carbon-carbon double bond of the tetrahydropyridines of formula XVIA.

Compounds of the formula XIV can then be treated with vinylstannanes of the formula VII 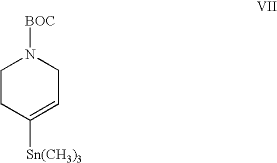
for example, 1-BOC-4-trimethylstannyl-1,2,5,6-tetrahydropyridine (wherein BOC refers to tert-butyloxycarbonyl), in the presence of a catalyst, to form the corresponding compound of formula XVIA. Palladium is the preferred catalyst (for example, ((C₆H₅)₃P)₄Pd or Pd₂(dba)₃), wherein dba refers to dibenzylidene acetone. Suitable solvents for the aforesaid reaction, when present, include acetonitrile, dimethylformamide, N-methyl-2-pyrrolidinone, preferably dimethylformamide. This reaction is conveniently run at about 20° C. to about 160° C., preferably about 60° C. to about 130° C. This reaction may be carried out as described in “Palladium-catalyzed Vinylation of Organic Halides” in Organic Reactions, 27, 345-390, (W. G. Dauben, Ed., John Wiley & Sons, Inc., New York, New York, 1982).

Compounds of the formula XVIA can be converted into compounds of the formula II, wherein R¹ is tetrahydropyridine by removal of the aldehyde or ketone-protecting group. The protecting group for the aldehyde or ketone, P, can be converted into the unprotected ketone or aldehyde of the formula —C(═O)R² using one or more of the techniques described in Greene, for example, stirring a solution of the compound of formula XVI in THF and 5% hydrochloric acid at room temperature for 20 hours.

Alternatively, compounds of formula XVIA can be converted into compounds of the formula II, where R¹ is piperidine (G²), by catalytic hydrogenation of the tetrahydropyridine of formula XVIA, from the previous paragraph, using standard methods known in the art, generally using palladium on carbon as the catalyst, to form the corresponding compounds of formula XVIB. This reaction is typically performed in an inert solvent, such as ethanol or ethyl acetate, either with or without a protic acid such as acetic acid or hydrochloric acid (HCl). Acetic acid is preferred. The protecting groups on G² (e.g., BOC) can be removed using one or more of the techniques described in Greene, referred to above, for example, stirring the compound of formula XVI in ethyl acetate and 3 molar hydrochloric acid at about room temperature for about 30 minutes. The protecting group for the aldehyde or ketone, P, can be converted into the unprotected ketone or aldehyde as described above.

Compounds of the formula XIV from reaction Scheme 2 may also be treated with alkyllithium reagents, for example n-butyllithium, sec-butyllithium or tert-butyllithium, preferably n-butyllithium in an inert solvent, as shown in Scheme 3, to form the intermediate lithium anion of formula XVII. Suitable solvents for this reaction include, for example, ether or tetrahydrofuran, preferably tetrahydrofuran. Reaction temperatures for this reaction can range from about −110° C. to about 0° C. The intermediate lithium anions of formula XVII can then be further reacted with a suitable electrophile, selection of which depends on the presence and nature of the substituent. Suitable electrophiles for use in preparing compounds of the formula II wherein R¹ is a group of the formula G² include, for example, carbonyl derivatives or alkylating agents (e.g., 1-BOC-4-piperidone). In the case where an aldehyde or ketone is used as the electrophile, the hydroxy group must be removed from the intermediate of formula XVIII, as depicted below, in order to form the corresponding compound of formula II.

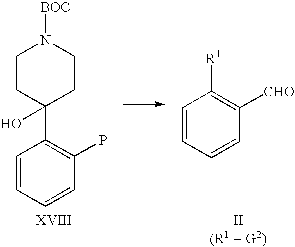

This step may be accomplished by one of several standard methods known in the art. For example, a thiocarbonyl derivative such as a xanthate may be prepared and removed by free radical processes, both of which are known to those skilled in the art. Alternatively, the hydroxyl group may be removed by reduction with a hydride source such as triethylsilane under acidic conditions, using, for example, trifluoroacetic acid or boron trifluoride. The reduction reaction can be performed neat or in a solvent such as methylene chloride. A further alternative would be to first convert the hydroxyl group to a suitable leaving group, such as tosylate or chloride, using standard methods known in the art, and then to remove the leaving group with a nucleophilic hydride, such as, for example, lithium aluminum hydride. The latter reaction is typically performed in an inert solvent such as ether or tetrahydrofuran. Also, a reducing agent may be used to reductively remove the benzylic substituent. Suitable reducing agents include, for example, Raney nickel in ethanol and sodium or lithium in liquid ammonia. Another alternative method for removing the hydroxyl group is to first dehydrate the alcohol of formula XVIII to an olefin of the formula XVIA (i.e. see Scheme 2) with a reagent such as Burgess salt (J. Org. Chem., 38, 26 (1973)) and then to catalytically hydrogenate the double bond under standard conditions with a catalyst such as palladium on carbon. The alcohol may also be dehydrated to the olefin by treatment with acids such as p-toluenesulfonic acid.

Compounds of the formula II, wherein R¹ is G² and R⁶ is hydrogen, can be converted into the corresponding compounds of the formula II, wherein R¹ is G² and R⁶ is other than hydrogen, by reacting them with a compound of the formula R⁶L′, as described above in Scheme 1, for preparing compounds of the formula VII.

Unless indicated otherwise, the pressure of each of the above reactions is not critical. Generally, the reactions will be conducted at a pressure of about one to about three atmospheres, preferably at ambient pressure (about one atmosphere).

The compounds of the formula I which are basic in nature are capable of forming a wide variety of different salts with various inorganic and organic acids. Although such salts must be pharmaceutically acceptable for administration to animals, it is often desirable in practice to initially isolate a compound of the formula I from the reaction mixture as a pharmaceutically unacceptable salt and then simply convert the latter back to the free base compound by treatment with an alkaline reagent, and subsequently convert the free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid addition salts of the base compounds of this invention are those which form non-toxic acid addition salts, i.e., salts containing pharmacologically acceptable anions, such as hydrochloride, hydrobromide, hydroiodide, nitrate, sulfate or bisulfate, phosphate or acid phosphate, acetate, lactate, citrate or acid citrate, tartrate or bitartrate, succinate, maleate, fumarate, gluconate, saccharate, benzoate, methanesulfonate and pamoate (i.e., 1,1′-methylene-bis-(2-hydroxy-3-naphthoate)) salts.

Those compounds of the formula I which are also acidic in nature, e.g., where R³ includes a COOH or tetrazole moiety, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the herein described acidic compounds of formula I. These non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum product yields.

Compounds of the formula I and their pharmaceutically acceptable salts (hereinafter also referred to, collectively, as “the active compounds”) are useful psychotherapeutics and are potent agonists and/or antagonists of the serotonin 1A (5-HT_{1A}) and/or serotonin 1B (5-HT_{1B}) receptors. The active compounds are useful in the treatment of hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders), bipolar disorder (including in the depressed phase), and attention-deficit/hyperactivity disorder (ADHD).

The affinities of the compounds of this invention for the various serotonin-1 receptors can be determined using standard radioligand binding assays as described in the literature. The 5-HT_{1A} affinity can be measured using the procedure of Hoyer et al. (Brain Res., 376, 85 (1986)). The 5-HT_{1B} affinity can be measured using the procedure of Heuring and Peroutka (J. Neurosci., 7, 894 (1987)).

The in vitro activity of the compounds of the present invention at the 5-HT_{1B} binding site may be determined according to the following procedure. Bovine caudate tissue is homogenized and suspended in 20 volumes of a buffer containing 50 mM TRIS.hydrochloride (tris[hydroxymethyl]aminomethane hydrochloride) at a pH of 7.7. The homogenate is then centrifuged at 45,000 G for 10 minutes. The supernatant is then discarded and the resulting pellet resuspended in approximately 20 volumes of 50 mM TRIS.hydrochloride buffer at pH 7.7. This suspension is then pre-incubated for 15 minutes at 37° C., after which the suspension is centrifuged again at 45,000 G for 10 minutes and the supernatant discarded. The resulting pellet (approximately 1 gram) is resuspended in 150 ml of a buffer of 15 mM TRIS.hydrochloride containing 0.01 percent ascorbic acid with a final pH of 7.7 and also containing 10 μM pargyline and 4 mM calcium chloride (CaCl₂). The suspension is kept on ice at least 30 minutes prior to use.

The inhibitor, control or vehicle is then incubated according to the following procedure. To 50 μl of a 20 percent dimethylsulfoxide (DMSO)/80 percent distilled water solution is added 200 μl of tritiated 5-hydroxytryptamine (2 nM) in a buffer of 50 mM TRIS.hydrochloride containing 0.01 percent ascorbic acid at pH 7.7 and also containing 10 μM pargyline and 4 μM calcium chloride, plus 100 nM of 8-hydroxy-DPAT (dipropylaminotetraline) and 100 nM of mesulergine. To this mixture is added 750 μl of bovine caudate tissue, and the resulting suspension is vortexed to ensure a homogenous suspension. The suspension is then incubated in a shaking water bath for 30 minutes at 25° C. After incubation is complete, the suspension is filtered using glass fiber filters (e.g., Whatman GF/B-filters™). The pellet is then washed three times with 4 ml of a buffer of 50 mM TRIS.hydrochloride at pH 7.7. The pellet is then placed in a scintillation vial with 5 ml of scintillation fluid (aquasol 2™) and allowed to sit overnight. The percent inhibition can be calculated for each dose of the compound. An IC₅₀ value can then be calculated from the percent inhibition values.

The activity of the compounds of the present invention for 5-HT_{1A} binding ability can be determined according to the following procedure. Rat brain cortex tissue is homogenized and divided into samples of one gram lots and diluted with 10 volumes of 0.32 M sucrose solution. The suspension is then centrifuged at 900 G for 10 minutes and the supernate separated and recentrifuged at 70,000 G for 15 minutes. The supernate is discarded and the pellet re-suspended in 10 volumes of 15 mM TRIS.hydrochloride at pH 7.5. The suspension is allowed to incubate for 15 minutes at 37° C. After pre-incubation is complete, the suspension is centrifuged at 70,000 G for 15 minutes and the supernate discarded. The resulting tissue pellet is resuspended in a buffer of 50 mM TRIS.hydrochloride at pH 7.7 containing 4 mM of calcium chloride and 0.01 percent ascorbic acid. The tissue is stored at −70° C. until ready for an experiment. The tissue can be thawed immediately prior to use, diluted with 10 μm pargyline and kept on ice.

The tissue is then incubated according to the following procedure. Fifty microliters of control, inhibitor, or vehicle (1 percent DMSO final concentration) is prepared at various dosages. To this solution is added 200 μl of tritiated DPAT at a concentration of 1.5 nM in a buffer of 50 mM TRIS.hydrochloride at pH 7.7 containing 4 mM calcium chloride, 0.01 percent ascorbic acid and pargyline. To this solution is then added 750 μl of tissue and the resulting suspension is vortexed to ensure homogeneity. The suspension is then incubated in a shaking water bath for 30 minutes at 37° C. The solution is then filtered, washed twice with 4 ml of 10 mM TRIS.hydrochloride at pH 7.5 containing 154 mM of sodium chloride. The percent inhibition is calculated for each dose of the compound, control or vehicle. IC₅₀ values are calculated from the percent inhibition values.

The compounds of formula I of the present invention described in the following Examples were assayed for 5-HT_{1A} and 5-HT_{1B} affinity using the aforementioned procedures. All such compounds of the invention that were tested exhibited IC₅₀'s less than 0.60 μM for 5-HT_{1B} affinity and IC₅₀'s less than 1.0 μM for 5-HT_{1A} affinity.

The agonist and antagonist activities of the compounds of the invention at 5-HT_{1A} and 5-HT_{1B} receptors can be determined using a single saturating concentration according to the following procedure. Male Hartley guinea pigs are decapitated and 5-HT_{1A} receptors are dissected out of the hippocampus, while 5-HT_{1B} receptors are obtained by slicing at 350 mM on a Mcllwain tissue chopper and dissecting out the substantia nigra from the appropriate slices. The individual tissues are homogenized in 5 mM HEPES buffer containing 1 mM EGTA (pH 7.5) using a hand-held glass-Teflon® homogenizer and centrifuged at 35,000×g for 10 minutes at 4° C. The pellets are resuspended in 100 mM HEPES buffer containing 1 mM EGTA (pH 7.5) to a final protein concentration of 20 mg (hippocampus) or 5 mg (substantia nigra) of protein per tube. The following agents are added so that the reaction mix in each tube contained 2.0 mM MgCl₂, 0.5 mM ATP, 1.0 mM cAMP, 0.5 mM IBMX, 10 mM phosphocreatine, 0.31 mg/mL creatine phosphokinase, 100 μM GTP and 0.5-1 microcuries of [³²P]-ATP (30 Ci/mmol: NEG-003—New England Nuclear). Incubation is initiated by the addition of tissue to siliconized microfuge tubes (in triplicate) at 30° C. for 15 minutes. Each tube receives 20 μL tissue, 10 μL drug or buffer (at 10× final concentration), 10 μL 32 nM agonist or buffer (at 10× final concentration), 20 μL forskolin (3 μM final concentration) and 40 μL of the preceding reaction mix. Incubation is terminated by the addition of 100 μL 2% SDS, 1.3 mM cAMP, 45 mM ATP solution containing 40,000 dpm [³H]-cAMP (30 Ci/mmol: NET-275—New England Nuclear) to monitor the recovery of cAMP from the columns. The separation of [³²P]-ATP and [³²P]-cAMP is accomplished using the method of Salomon et al., Analytical Biochemistry, 1974, 58, 541-548. Radioactivity is quantified by liquid scintillation counting. Maximal inhibition is defined by 10 μM (R)-8-OH-DPAT for 5-HT_{1A} receptors, and 320 nM 5-HT for 5-HT_{1B} receptors. Percent inhibitions by the test compounds are then calculated in relation to the inhibitory effect of (R)-8-OH-DPAT for 5-HT_{1A} receptors or 5-HT for 5-HT_{1B} receptors. The reversal of agonist induced inhibition of forskolin-stimulated adenylate cyclase activity is calculated in relation to the 32 nM agonist effect.

The compounds of the invention can be tested for in vivo activity for antagonism of 5-HT_{1B} agonist-induced hypothermia in guinea pigs according to the following procedure.

Male Hartley guinea pigs from Charles River, weighing 250-275 grams on arrival and 300-600 grams at testing, serve as subjects in the experiment. The guinea pigs are housed under standard laboratory conditions on a 7 a.m. to 7 p.m. lighting schedule for at least seven days prior to experimentation. Food and water are available ad libitum until the time of testing.

The compounds of the invention can be administered as solutions in a volume of 1 ml/kg. The vehicle used is varied depending on compound solubility. Test compounds are typically administered either sixty minutes orally (p.o.) or 0 minutes subcutaneously (s.c.) prior to a 5-HT_{1B} agonist, such as [3-(1-methylpyrrolidin-2-ylmethyl)-1H-indol-5-yl]-(3-nitropyridin-3-yl)-amine, which can be prepared as described in PCT publication WO93/11106, published Jun. 10, 1993 which is administered at a dose of 5.6 mg/kg, s.c. Before a first temperature reading is taken, each guinea pig is placed in a clear plastic shoe box containing wood chips and a metal grid floor and allowed to acclimate to the surroundings for 30 minutes. Animals are then returned to the same shoe box after each temperature reading. Prior to each temperature measurement each animal is firmly held with one hand for a 30-second period. A digital thermometer with a small animal probe is used for temperature measurements. The probe is made of semi-flexible nylon with an epoxy tip. The temperature probe is inserted 6 cm. into the rectum and held there for 30 seconds or until a stable recording is obtained. Temperatures are then recorded.

In p.o. screening experiments, a “pre-drug” baseline temperature reading is made at −90 minutes, the test compound is given at −60 minutes and an additional −30 minute reading is taken. The 5-HT_{1B} agonist is then administered at 0 minutes and temperatures are taken 30, 60, 120 and 240 minutes later.

In subcutaneous screening experiments, a pre-drug baseline temperature reading is made at −30 minutes. The test compound and 5-HT_{1B} agonists are given concurrently and temperatures are taken at 30, 60, 120 and 240 minutes later.

Data are analyzed with two-way analysis of variants with repeated measures in Newman-Keuls post hoc analysis.

The active compounds of the invention can be evaluated as anti-migraine agents by testing the extent to which they mimic sumatriptan in contracting the dog isolated saphenous vein strip (P. P. A. Humphrey et al., Br. J. Pharmacol., 94, 1128 (1988)). This effect can be blocked by methiothepin, a known serotonin antagonist. Sumatriptan is known to be useful in the treatment of migraine and produces a selective increase in carotid vascular resistance in the anesthetized dog. The pharmacological basis of sumatriptan efficacy has been discussed in W. Fenwick et al., Br. J. Pharmacol., 96, 83 (1989).

The serotonin 5-HT₁ agonist activity can be determined by the in vitro receptor binding assays, as described for the 5-HT_{1A} receptor using rat cortex as the receptor source and [³H]-8-OH-DPAT as the radioligand (D. Hoyer et al. Eur. J. Pharm., 118, 13 (1985)) and as described for the 5-HT_{1B} receptor using bovine caudate as the receptor source and [³H]serotonin as the radioligand (R. E. Heuring and S. J. Peroutka, J. Neuroscience, 7, 894 (1987)). Of the active compounds tested, all exhibited an IC₅₀ in either assay of 1 μM or less.

The compounds of formula I may advantageously be used in conjunction with one or more other therapeutic agents, for instance, different antidepressant agents such as tricyclic antidepressants (e.g., amitriptyline, dothiepin, doxepin, trimipramine, butripyline, clomipramine, desipramine, imipramine, iprindole, lofepramine, nortriptyline or protriptyline), monoamine oxidase inhibitors (e.g., isocarboxazid, phenelzine or tranylcyclopramine) or 5-HT re-uptake inhibitors (e.g., fluvoxamine, sertraline, fluoxetine or paroxetine), and/or with antiparkinsonian agents such as dopaminergic antiparkinsonian agents (e.g., levodopa, preferably in combination with a peripheral decarboxylase inhibitor e.g., benserazide or carbidopa, or with a dopamine agonist e.g., bromocriptine, lysuride or pergolide). It is to be understood that the present invention covers the use of a compound of general formula (I) or a physiologically acceptable salt or solvate thereof in combination with one or more other therapeutic agents.

Compounds of the formula I and the pharmaceutically acceptable salts thereof, in combination with a 5-HT re-uptake inhibitor (e.g., fluvoxamine, sertraline, fluoxetine or paroxetine), preferably sertraline, or a pharmaceutically acceptable salt or polymorph thereof (the combination of a compound of formula I with a 5-HT re-uptake inhibitor is referred herein to as “the active combination”), are useful psychotherapeutics and may be used in the treatment of disorders the treatment of which is facilitated by enhanced serotonergic neurotransmission (e.g., hypertension, all forms of depression (e.g., depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition), generalized anxiety disorder, phobias (e.g., agoraphobia, social phobia and simple phobias), posttraumatic stress syndrome, avoidant personality disorder, premature ejaculation, eating disorders (e.g., anorexia nervosa and bulimia nervosa), obesity, chemical dependencies (e.g., addictions to alcohol, cocaine, heroin, phenobarbital, nicotine and benzodiazepines), cluster headache, migraine, pain, Alzheimer's disease, obsessive-compulsive disorder, panic disorder, memory disorders (e.g., dementia, amnestic disorders, and age-related cognitive decline (ARCD)), Parkinson's diseases (e.g., dementia in Parkinson's disease, neuroleptic-induced parkinsonism and tardive dyskinesias), endocrine disorders (e.g., hyperprolactinaemia), vasospasm (particularly in the cerebral vasculature), cerebellar ataxia, gastrointestinal tract disorders (involving changes in motility and secretion), negative symptoms of schizophrenia, premenstrual syndrome, fibromyalgia syndrome, stress incontinence, Tourette's syndrome, trichotillomania, kleptomania, male impotence, cancer (e.g. small cell lung carcinoma), chronic paroxysmal hemicrania, headache (associated with vascular disorders), bipolar disorder (including in the depressed phase), and attention-deficit/hyperactivity disorder (ADHD).

Serotonin (5-HT) re-uptake inhibitors, preferably sertraline, exhibit positive activity against depression; chemical dependencies; anxiety disorders including panic disorder, generalized anxiety disorder, agoraphobia, simple phobias, social phobia, and post-traumatic stress disorder; obsessive-compulsive disorder; avoidant personality disorder and premature ejaculation in mammals, including humans, due in part to their ability to block the synaptosomal uptake of serotonin.

U.S. Pat. No. 4,536,518 describes the synthesis, pharmaceutical composition and use of sertraline for depression and is hereby incorporated by reference in its entirety.

Activity of the active combination as antidepressants and related pharmacological properties can be determined by methods (1)-(4) below, which are described in Koe, B. et al., Journal of Pharmacology and Experimental Therapeutics, 226 (3), 686-700 (1983). Specifically, activity can be determined by studying (1) their ability to affect the efforts of mice to escape from a swim-tank (Porsolt mouse “behavior despair” test), (2) their ability to potentiate 5-hydroxytryptophan-induced behavioral symptoms in mice in vivo, (3) their ability to antagonize the serotonin-depleting activity of p-chloroamphetamine hydrochloride in rat brain in vivo, and (4) their ability to block the uptake of serotonin, norepinephrine and dopamine by synaptosomal rat brain cells in vitro. The ability of the active combination to counteract reserpine hypothermia in mice in vivo can be determined according to the methods described in U.S. Pat. No. 4,029,731.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insulator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is 0.1 to 200 mg of the active ingredient per unit dose which could be administered, for example, 1 to 4 times per day.

Aerosol formulations for treatment of the conditions referred to above (e.g., migraine) in the average adult human are preferably arranged so that each metered dose or “puff” of aerosol contains 20 μg to 1000 μg of the compound of the invention. The overall daily dose with an aerosol will be within the range 100 μg to 10 mg. Administration may be several times daily, for example 2, 3, 4 or 8 times, giving for example, 1, 2 or 3 doses each time.

In connection with the use of an active compound of this invention with a 5-HT re-uptake inhibitor, preferably sertraline, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 90% by weight of the total composition, i.e., in amounts which are sufficient to provide the desired unit dosage and a 5-HT re-uptake inhibitor, preferably sertraline, is present in such dosage forms at concentration levels ranging from about 0.5% to about 90% by weight of the total composition, i.e., in amounts which are sufficient to provide the desired unit dosage.

A proposed daily dose of an active compound of this invention in the combination formulation (a formulation containing an active compound of this invention and a 5-HT re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration may be several times daily, for example 2, 3, 4 or 8 times, giving for example, 1, 2 or 3 doses each time.

As previously indicated, a 5-HT re-uptake inhibitor, preferably sertraline, in combination with compounds of formula I are readily adapted to therapeutic use as antidepressant agents. In general, these antidepressant compositions containing a 5-HT re-uptake inhibitor, preferably sertraline, and a compound of formula I are normally administered in dosages ranging from about 0.01 mg to about 100 mg per kg of body weight per day of a 5-HT re-uptake inhibitor, preferably sertraline, preferably from about 0.1 mg. to about 10 mg per kg of body weight per day of sertraline; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

### EXAMPLES

The following Examples illustrate the preparation of the compounds of the present invention. Melting points are uncorrected. NMR data are reported in parts per million (δ) and are referenced to the deuterium lock signal from the sample solvent (deuteriochloroform unless otherwise specified). Specific rotations were measured at room temperature using the sodium D line (589 nm). Commercial reagents were utilized without further purification. THF refers to tetrahydrofuran. DMF refers to N,N-dimethylformamide. Chromatography refers to column chromatography performed using 32-63 mm silica gel and executed under nitrogen pressure (flash chromatography) conditions. Room or ambient temperature refers to 20-25° C. All non-aqueous reactions were run under a nitrogen atmosphere for convenience and to maximize yields. Concentration at reduced pressure means that a rotary evaporator was used.

### Example 1

### 2-[2-(4-Methylpiperazin-1-yl)-benzylidene]-4-(4-trifluoromethylphenyl)-morpholin-3-one

Under N2 atmosphere, a slurry of NaH (0.118 g, 2.94 mmol of a 60% dispersion in oil) and 6 mL anhydrous THF was treated portionwise with 0.199 g (0.98 mmol) of 2-(4-methylpiperazin-1-yl)-benzaldehyde, 0.300 g (1.22 mmol, see Preparation 1) and 4 mL THF with some foaming. The mixture was stirred at room temperature for 90 min. and then heated to reflux. After five days, the reaction was cooled to room temperature and the mixture quenched by adding water, then ethyl acetate, producing a thick emulsion. This was made acidic with 2N HCl, and extracted with Et₂O. The aqueous layer was then made basic with 2N NaOH and re-extracted with methylene chloride. The organic extracts were washed with water and saturated aqueous NaCl, dried with MgSO₄ and concentrated in vacuo to a tan oil, 0.161 g. Mass spectrum 431 (M+). ¹H-NMR (CDCl₃, 250 MHz) δ 8.04 (1H, dd), 7.65 (2H, m), 7.53 (2H, m), 7.23 (2H, m), 7.05 (2H, m), 4.39 (2H, m), 4.01 (2H, m), 3.06 (4H,bs), 2.72 (4H, bs), 2.39 (3H, s).

The following compounds in Examples 2-22 were made in a similar manner as described in Example 1:

### Example 2

### 2-[2-(4-Methylpiperazin-1-yl)-benzylidene]-4-(3-trifluoromethylphenyl)-morpholin-3-one hydrochloride

White solid. Mass spectrum 432 (M+1).

¹H-NMR (DMSO-d₆, 250 MHz) δ 7.97 (1H, d), 7.89 (1H, s), 7.60 (2H, m), 7.23 (1H, dd), 7.07 (2H, m), 7.01 (1H, s), 4.37 (2H, m), 4.05 (2H, m), 3.43 (2H, m), 3.12 (4H, m), 3.00 (2H, m), 2.80 (3H, s).

### Example 3

### 2-[2-(4-Methylpiperazin-1-yl)-benzylidene]-4-(4-isopropylphenyl)-morpholin-3-one

Solid, M.P. 155-156° C. Mass spectrum 406 (M+1).

¹H-NMR (CDCl₃, 250 MHz) δ 8.05 (1H, dd), 7.33 (1H, s), 7.24 (4H, m), 7.03 (2H, m), 4.36 (2H, m), 3.96 (2H, m), 3.10 (4H, bs), 2.89 (1H, m), 2.75 (4H, bs), 2.36 (3H, bs), 1.25 (6H, d).

### Example 4

### 2-[2-(4-Methylpiperazin-1-yl)-benzylidene]-4-phenyl-morpholin-3-one

Light brown oil. Mass spectrum 364 (M+1).

### Example 5

### 2-[2-(4-Methylpiperazin-1-yl)-benzylidene]-4-(4-tert-butylphenyl)-morpholin-3-one

Off-white solid. M.P. 169-171° C. Mass spectrum 420 (M+1).

¹H-NMR (CDCl₃, 250 MHz) δ 8.06 (1H, dd), 7.41 (2H, m), 7.39 (1H, s), 7.28 (2H, m), 7.19 (2H, m), 7.02 (2H, m), 4.35 (2H, m), 3.95 (2H, m), 3.00 (4H, bs), 2.63 (4H, bs), 2.32 (3H, s), 1.29 (9H, s).

Elemental analysis calculated for C₂₆H₃₃N₃O₂.H₂O: C, 71.37, H, 8.06, N, 9.60. Found: C, 71.73, H, 7.92, N, 9.34

### Example 6

### 2-[2-(3,4,5-Trimethylpiperazin-1-yl)-benzylidene]-4-(4-tert-butylphenyl)-morpholin-3-one

Light tan solid. M.P. 167-168° C. Mass spectrum 448 (M+1).

### Example 7

### 4-[4-(1-Hydroxy-1-methylethyl)-phenyl-2-[2-(4-methylpiperazin-1-yl)-benzylidene]-morpholin-3-one

Tan oil. Mass spectrum 422 (M+1).

¹H-NMR (CDCl₃, 250 MHz) δ 8.05 (1 H, dd), 7.51 (2H, d), 7.33 (3H, m), 7.20 (1H, m), 7.01 (2H, m), 4.35 (2H, m), 3.95 (2H, m), 3.00 (4H, bs), 2.65 (4H, bs), 2.33 (3H, s), 1.55 (6H, s).

### Example 8

### 4-(4-tert-Butyl-phenyl)-2-[4-fluoro-2-(4-methylpiperazin-1-yl)-benzylidene]-morpholin-3-one

Light yellow oil. Mass spectrum 438 (M+1).

¹H-NMR (CDCl₃, 250 MHz) δ 7.82 (1H, dd), 7.38 (3H, m), 7.28 (2H, m), 6.94 (1H, m), 6.90 (1 H, dt), 4.37 (2H, m), 3.95 (2H, m), 2.95 (4H, bs), 2.58 (4H, bs), 2.28 (3H, s), 1.29 (9H, 2).

### Example 9

### 4-(4-tert-Butyl-phenyl)-2-[6-chloro-2-(4-methylpiperazin-1-yl)-benzylidene]-morpholin-3-one hydrochloride

White solid. M.P. 139-142° C. Mass spectrum 454 (M+), 456.

¹H-NMR (CD₃OD 250 MHz) δ 7.48 (2H, dd), 7.33 (2H, d), 7.26 (1H, t), 7.20 (1H, m), 7.05 (1H, dd), 6.84 (1H, s), 4.31 (2 H, m), 3.95 (2 H, m), 3.53 (2 H, m), 3.26 (4 H, m), 3.08 (2 H, t), 2.93 (3 H, s), 1.32 (9 H, s).

### Example 10

### 4-(4-tert-Butyl-phenyl)-2-[4-chloro-2-(4-methylpiperazin-1-yl)-benzylidene]-morpholin-3-one

Beige solid. M.P. 209-210.8° C. Mass spectrum 454 (M+), 456.

¹H-NMR (DMSO-d₆, 250 MHz) δ 7.93 (1H, d), 7.39 (2H, d), 7.30 (2H, d), 7.05 (1H, dd), 6.98 (1H, d), 6.93 (1H, s), 4.34 (2H, dt), 3.94 (2H, dt), 3.26 (4H, s+m), 2.84 (4H, bs), 2.17 (3H, bs), 1.25 (9H, s).

### Example 11

### 4-(4-tert-Butyl-phenyl)-2-[6-fluoro-2-(4-methylpiperazin-1-yl)-benzylidene]-morpholin-3-one hydrochloride

Pale yellow solid. M.P. 187.5-192.6° C. Mass spectrum 438 (M+1).

¹H-NMR (CD₃OD, 250 MHz) δ 7.48 (2H, dd), 7.32 (2H, d), 6.90 (2H, dd), 6.77 (1H, s), 4.30 (2H, m), 3.95 (2H, m), 3.54 (2H, d), 3.37 (2H, m), 3.27 (2H, m), 3.06 (2H, t), 2.93 (3H, s), 1.31 (9H, s).

### Example 12

### 4-(4-tert-Butyl-phenyl)-2-[3-fluoro-2-(4-methylpiperazin-1-yl)-benzylidene]-morpholin-3-one

Tan solid. M.P. 184.3-187.1° C. Mass spectrum 438 (M+1).

### Example 13

### 4-(4-tert-Butyl-phenyl)-2-[2-(4-methylpiperazin-1-yl)-6-trifluoromethyl-benzylidene]-morpholin-3-one hydrochloride

Off-white solid. M.P. 205° C. dec. Mass spectrum 488 (M+1).

¹H-NMR (CD₃OD, 250 MHz) δ 7.47 (4H, m), 7.41 (1H, m), 7.34 (2H, m), 6.91 (1H, m), 4.21 (2H, dd), 3.93 (2H, dd), 3.56 (2H, bd), 3.28 (3H, d), 3.27 (1H, m), 3.26 (2H, m), 2.91 (2H, s), 1.31 (9H, s).

### Example 14

### 4-(4-tert-Butyl-phenyl)-2-[2-(4-methylpiperazin-1-yl)-4-trifluoromethyl-benzylidene]-morpholin-3-one

Light brown solid. M.P. 186.3-191.9° C. Mass spectrum 488 (M+1).

¹H-NMR (CDCl₃, 250 MHz) δ 8.14 (1H, dd), 7.43 (2H, m), 7.34 (1H, m), 7.26 (4H, m), 4.42 (2H, m), 4.00 (2H, m), 3.59 (2H, t), 3.50 (2H, d), 3.23 (2H, d), 3.12 (2H, m), 2.78 (3H, s).

### Example 15

### 4-(4-tert-Butyl-phenyl)-2-[5-methyl-2-(4-methylpiperazin-1-yl)-benzylidene]-morpholin-3-one

Light tan solid. M.P. 209.2-211.0° C. Mass spectrum 434 (M+1).

¹H-NMR (CDCl₃, 250 MHz) δ 7.86 (1H, s), 7.41 (2H, d), 7.39 (1H, s), 7.28 (2H, d), 7.03 (1H, dd), 6.92 (1H, d), 4.36 (2H, m), 3.97 (2H, m), 3.04 (4H, bs), 2.74 (4H, bs), 2.39 (3H, s), 2.30 (3H, s), 1.29 (9H, s).

### Example 16

### 4-(4-tert-Butyl-phenyl)-2-[2-(3-(R)-dimethylamino-pyrrolidin-1-yl)-benzylidene]-morpholin-3-one

Tan solid. Mass spectrum 434 (M+1).

¹H-NMR (CDCl₃, 250 MHz) δ 7.85 (1H, dd), 7.40 (2H, dd), 7.28 (1H, m), 7.14 (2H, m), 6.89 (2H, m), 4.33 (2H, m), 3.94 (2H, m), 3.22 (5H, m), 2.98 (1H, m), 2.28 (6H, s), 2.10 (1H, m), 1.84 (1H, m), 1.29 (9H, s).

### Example 17

### 4-(4-tert-Butyl-phenyl)-2-[2-(4-methylpiperazin-1-yl)-5-trifluoromethyl-benzylidene]-morpholin-3-one

Yellow solid. Mass spectrum 488 (M+1).

### Example 18

### 4-Biphenyl-4-yl-2-[2-(4-methylpiperazin-1-yl)-benzylidene]-morpholin-3-one

Amber solid. Mass spectrum 440 (M+1).

### Example 19

### 4-Benzyl-2-[2-(4-methylpiperazin-1-yl)-benzylidene]-morpholin-3-one

Tan solid. Mass spectrum 378 (M+1).

### Example 20

### 4-(4-tert-Butyl-benzyl)-2-[2-(4-methylpiperazin-1-yl)-benzylidene]-morpholin-3-one

White foam. M.P. 68-70° C. Mass spectrum 434 (M+1).

¹H-NMR (CDCl₃, 250 MHz) δ 7.98 (1 H, dd), 7.34 (2H, m), 7.22 (4H, m), 7.03 (2H, m), 4.67 (2H, s), 4.15 (2H, m), 3.47 (2H, m), 3.12 (6H, m), 2.67 (3H, bs), 1.50 (2H, bs), 1.28 (9H, s).

### Example 21

### 4-(4-Chlorobenzyl)-5-methyl-2-[2-(4-methylpiperazin-1-yl)-benzylidene]-morpholin-3-one hydrochloride

White solid. Mass spectrum 426 (M+1).

¹H-NMR (DMSO-d₆, 250 MHz) δ 7.97 (1H, dd), 7.39 (2H, m), 7.33 (2H, m), 7.25 (1H, m), 7.08 (2H, m), 6.93 (1H, s), 4.92 (1H, dd), 4.33 (1H, dd), 4.16 (2H, dq), 3.64 (1H, m), 3.58 (2H, m), 3.31 (4H, m), 3.27 (2H, m), 2.86 (3H, s), 1.23 (3H, d).

### Example 22

### 2-[2-(4-Methylpiperazin-1-yl)-benzylidene]-4-pyridin-3-yl-morpholin-3-one

Tan, waxy solid. Mass spectrum 365 (M+1).

¹ H-NMR (CDCl₃, 250 MHz) δ 8.64 (1H, d), 8.46 (1H, dd), 8.03 (1H, dd), 7.81 (1H, m), 7.38 (1H, s), 7.33 (1H, m), 7.22 (1H, m), 7.03 (2H, m), 4.37 (2H, m), 4.00 (2H, m), 2.97 (4H, m), 2.59 (4H, bs), 2.30 (3H, s).

### Example 23

### (±)-4-(4-tert-Butyl-phenyl)-2-[2-(4-methylpiperazin-1-yl)-benzyl]-morpholin-3-one

Method A.

Under N2, a slurry of 10% Pd on carbon (1.5 g, Aldrich Chemical Company) in 300 mL absolute ethanol was treated with 2-[2-(4-methylpiperazin-1-yl)-benzylidene]4-(4-tert-butylphenyl)-morpholin-3-one (9.0 g, 21.5 mmol; the title compound of Example 5) at room temperature, followed by ammonium formate (13.55 g, 214.8 mmol). After 90 min. the temperature was increased to 50-55° C. and maintained at this level for 18 hr. After cooling to room temperature, the mixture was filtered through d.e., washing the pad with several portions of ethanol and water. The filtrates were concentrated in vacuo to a white foam, which was partitioned between ethyl acetate and saturated aqueous Na₂CO₃, the aqueous layer re-extracted with additional portions of ethyl acetate, and the combined organic extracts washed with water and saturated NaCl. After drying with MgSO₄, the solvent was removed in vacuo to give an off-white solid, 7.0 g. M.P. 149.2-150.1° C. Mass spectrum 422 (M+1).

A mixture of the preceding free base (0.14 g, 0.277 mmol) in 3 mL of isopropanol was treated with (+)-L-tartaric acid (0.042 g, 0.277 mmol) and heated to boiling, then allowed to cool. The solvent was removed in vacuo, the residue recrystallized from 4.0 mL of hot methyl ethyl ketone (MEK) to give the hemi-tartrate salt as a white solid, 94 mg. M.P. 119-120.6° C.

The free base (0.153 g) was also converted with (−)-D-tartaric acid to the corresponding hemi-tartrate salt as a white solid, 172 mg, M.P. 115.1-116.9° C.

Method B.

In a 100 mL glass Parr shaker bottle, dissolved 2-[2-(4-methylpiperazin-1-yl)-benzylidene]-4-(4-tert-butylphenyl)-morpholin-3-one (0.20 g, 0.48 mmol) in 20 mL of absolute ethanol, then added 200 mg of 10% Pd on carbon. The bottle was placed on Parr Shaker hydrogenation apparatus and charged with hydrogen gas at 40 psig, then shaken for six days, occasionally adding fresh catalyst (100 mg portions) and recharging with H₂ gas. When the mass spectrum indicated the conversion to the desired product, the mixture was filtered under N₂ through a d.e. pad, the filter washed with additional ethanol and the filtrate concentrated in vacuo to a yellow oil. The oil was diluted with Et₂O and treated with 1.ON HCl in Et₂O at room temperature with stirring to give after one hr the title product as the hydrochloride salt. White solid, 93 mg. M.P 179-180° C. dec. Mass spectrum 422 (M+1).

Elemental Analysis calculated for C₂₆H₃₅N₃O₂.HCl.2H₂O: C, 63.21, H, 8.16, N, 8.50. Found: C, 63.19, H, 7.59, N, 8.19.

Method C.

A slurry of 2-[2-(4-methylpiperazin-1-yl)-benzylidene]-4-(4-tert-butylphenyl)-morpholin-3-one (0.40 g, 0.95 mmol) in 3 mL of anhydrous methanol (20 ml) was treated with samarium (II) iodide (30 ml of 0.1M SmI₂ in THF, Aldrich Chemical Co., Milwaukee, Wis.) and stirred 5 hr at room temperature under a nitrogen atmosphere. The mixture was then diluted with 10 mL water, the solvent was removed in vacuo and the residue was flash chromatographed using ethyl acetate/methanol to elute the free base of the product.

### Example 24

### Chiral separation of racemic (±)-4-(4-tert-Butyl-phenyl)-2-[2-(4-methylpiperazin-1-yl)-benzyl]-morpholin-3-one into the (+) and (−) enantiomers

A 3.98 g sample of the racemic compound made in Example 23A was separated using a preparatory HPLC method (10 cm×50 cm Chiralcel OD column, 275 mL/min flow rate, eluting with 5% ethanol in heptanes).

Fractions containing the first enantiomer (retention time of 10.7 min, 100% e.e.) were concentrated in vacuo to a pale brown oil, 1.88 g. The oil was dissolved in ethyl acetate and treated with an equivalent amount of ethyl acetate saturated with HCl gas. After 18 hr, the precipitated solids were filtered and washed with Et₂O and dried in vacuo to give 1.11 g of (+)-4-(4-tert-Butyl-phenyl)-2-[2-(4-methylpiperazin-1-yl)-benzyl]-morpholin-3-one mono-hydrochloride salt. M.P. 165.8-171.2° C. Mass spectrum 422 (M+1). [α]²⁵_{D}=+75.3° (c=1, MeOH).

Fractions containing the second, more polar enantiomer (retention time of 14.6 min, 98% e.e.) were concentrated in vacuo to a pale brown-oil, 2.30 g, and converted in the same manner as above to give 1.67 g of (−)-4-(4-tert-Butyl-phenyl)-2-[2-(4-methylpiperazin-1-yl)-benzyl]-morpholin-3-one monohydrochloride salt. M.P. 187.1-191.2° C. [a]²⁵_{D}=−85.9° (c=1, MeOH).

The following compounds of examples 25-38 were prepared in a similar manner:

### Example 25

### (±)-4-(4-Isopropyl-phenyl)-2-[2-(4-methylpiperazin-1-yl)-benzyl]-morpholin-3-one hydrochloride (Method A)

Off-white solid. M.P. 185.7-188.1° C. Mass spectrum 408 (M+1).

¹H-NMR (CD₃OD, 250 MHz) δ 7.41 (1H, dd), 7.24 (4H, m), 7.14 (3H, m), 4.52 (1H, dd), 4.10 (1H, m), 3.56 (2H, dt+m), 3.51 (4H, m), 3.26 (7H, m), 2.91 (3H, s), 2.90 (1H, m), 1.22 (6H, s).

Elemental analysis calculated for C₂₅H₃₃N₃O₂.HCl.3H₂O: C, 60.29, H,8.10, N, 8.44. Found: C, 60.76, H, 7.60, N, 8.50.

### (−)-4-(4-Isopropyl-phenyl)-2-[2-(4-methylpiperazin-1-yl)-benzyl]-morpholin-3-one citrate

White solid. M.P. 73-74° C. [α]²⁵_{D}=−66.93° (c=0.69, MeOH)

Elemental analysis calculated forC₂₅H₃₃N₃O₂.C₆H₈O₇.2H₂O: C, 58.57, H, 7.14, N, 6.61. Found: C, 58.71, H, 7.21, N, 6.55.

### (+)-4-(4-Isopropyl-phenyl)-2-[2-(4-methylpiperazin-1-yl)-benzyl]-morpholin-3-one citrate

White solid. M.P. 73-74° C.

Elemental analysis calculated forC₂₅H₃₃N₃O₂.C₆H₈N₇.2.5H₂O: C, 57.75, H, 7.19, N, 6.52. Found: C, 58.05, H, 7.25, N, 6.23.

### Example 26

### (±)-4-(4-tert-Butyl-phenyl)-2-[2-chloro-6-(4-methylpiperazin-1-yl)-benzyl]-morpholin-3-one hydrochloride (Method C)

Tan solid. M.P. 215° C. dec. Mass spectrum 456 (M+).

¹H-NMR (CD₃OD, 250 MHz) δ 7.48 (2H, d), 7.24 (2H, d), 7.22 (3H, m), 4.65 (1H, dd), 4.15 (1H, m), 3.84 (2H, m), 3.71 (1H, m), 3.48 (4H, m), 3.27 (4H, m), 3.10 (1H, dt), 2.92 (3H, s), 1.30 (9H, s).

### (+)-4-(4-tert-Butyl-phenyl)-2-[2-chloro-6-(4-methylpiperazin-1-yl)-benzyl]-morpholin-3-one hydrochloride

White solid. M.P. 107-109° C.

[α]²⁵_{D}=+33.3° (c=0.66, MeOH).

### (−)-4-(4-tert-Butyl-phenyl)-2-[2-chloro-6-(4-methylpiperazin-1-yl)-benzyl]-morpholin-3-one hydrochloride

White solid. M.P. 107-109° C.

[α]²⁵_{D}=−41.5° (c=0.53, MeOH).

### Example 27

### (±)-4-(4-tert-Butyl-phenyl)-2-[4-chloro-2-(4-methylpiperazin-1-yl)-benzyl]-morpholin-3-one (Method C)

Off-white solid. M.P. 161.9-163.5° C. Mass spectrum 456 (M+), 458.

### (+)-4-(4-tert-Butyl-phenyl)-2-[4-chloro-2-(4-methylpiperazin-1-yl)-benzyl]-morpholin-3-one hydrochloride

Pale yellow solid. M.P. 159.5-161.7° C.

[α]²⁵_{D}=+78.30 (c=0.54, MeOH).

### (−)-4-(4-tert-Butyl-phenyl)-2-[4-chloro-2-(4-methylpiperazin-1-yl)-benzyl]-morpholin-3-one hydrochloride

Off-white solid. M.P. 159-161.8° C.

[α]²⁵_{D}=−79.1° (c=0.52, MeOH).

### Example 28

### (±)-4-(4-tert-Butyl-phenyl)-2-[2-fluoro-6-(4-methylpiperazin-1-yl)-benzyl]-morpholin-3-one hydrochloride (Method C)

White solid. M.P. 190.5-192.1° C. Mass spectrum 440 (M+1).

¹H-NMR (CD₃OD, 250 MHz) δ 7.47 (2H, d), 7.27 (3H, m), 7.05 (1H, d), 6.92 (1H, t), 4.57 (1H, dd), 4.09 (1H, m), 3.84 (2H, m), 3.68 (1H, m), 3.56 (2H, m), 3.32 (6H, m), 3.18 (1H, m), 2.96 (1H, m), 2.93 (3H, s), 1.31 (9H, s).

### (+)-4-(4-tert-Butyl-phenyl)-2-[2-fluoro-6-(4-methylpiperazin-1-yl)-benzyl]-morpholin-3-one hydrochloride

White solid. M.P. 176.9-179.3° C.

Elemental analysis calculated for C₂₆H₃₄FN₃O₂.HCl.1.5H₂O: C, 57.88, H, 7.29, N, 7.79. Found: C, 57.90, H, 7.41, N, 7.44.

[α]²⁵_{D}=+40.00 (c=0.68, MeOH).

### (−)-4-(4-tert-Butyl-phenyl)-2-[2-fluoro-6-(4-methylpiperazin-1-yl)-benzyl]-morpholin-3-one hydrochloride

White solid. M.P. 178-181.4° C.

Elemental analysis calculated for C₂₆H₃₄FN₃O₂.HCl.3H₂O: C, 58.91, H, 7.80, N, 7.93. Found: C, 59.00, H, 7.28, N, 7.36.

[α]²⁵_{D}=−64.5° (c=0.65, MeOH).

### Example 29

### (±)-4-(4-tert-Butyl-phenyl)-2-[4-fluoro-2-(4-methylpiperazin-1-yl)-benzyl]-morpholin-3-one hydrochloride (Method A)

White solid. M.P. 155.9-159.9° C. Mass spectrum 440 (M+1).

¹H-NMR (CD₃OD, 250 MHz) δ 7.45 (2H, dd), 7.24 (1H, dd), 7.17 (3H, m), 6.98 (1H, dt), 4.51 (1H, dd), 4.10 (1H, dt), 3.92 (1H, dt), 3.84 (1H, dt), 3.60 (1H, m), 3.47 (3H, m), 3.28 (3H, m), 3.12 (5H, m), 1.30 (9H, s).

Elemental analysis calculated for C₂₆H₃₄FN₃O₂.HCl.2.5H₂O: C, 59.93, H, 7.74, N, 8.06. Found: C, 59.95, H, 8.19, N, 7.97.

### (+)-4-(4-tert-Butyl-phenyl)-2-[4-fluoro-2-(4-methylpiperazin-1-yl)-benzyl]-morpholin-3-one hydrochloride

White solid. M.P. 157.4-159.6° C. Mass spectrum 440 (M+1).

Elemental analysis calculated for C₂₆H₃₄FN₃O₂.HCl.3.5H₂O: C, 57.93, H, 7.85, N, 7.79. Found: C, 57.76, H, 7.32, N, 7.79.

[α]²⁵_{D}=+80.1° (c=1.11, MeOH).

### (−)-4-(4-tert-Butyl-phenyl)-2-[4-fluoro-2-(4-methylpiperazin-1-yl)-benzyl]-morpholin-3-one hydrochloride

White solid. M.P. 156.2-158.1° C. Mass spectrum 440 (M+1).

Elemental analysis calculated for C₂₆H₃₄FN₃O₂.HCl.3.5H₂O: C, 57.93, H, 7.85, N, 7.79. Found: C, 57.89, H, 7.88, N, 7.44.

[α]²⁵_{D}=−88.9° (c=1.11, MeOH).

### Example 30

### (±)-4-(4-tert-Butyl-phenyl)-2-[3-fluoro-2-(4-methylpiperazin-1-yl)-benzyl]-morpholin-3-one hydrochloride (Method C)

Off-white solid. M.P. 107-108.7° C. Mass spectrum 440 (M+1).

¹H-NMR (DMSO-d₆, 250 MHz) δ 7.38 (2H, dd), 7.21 (2H, m), 7.16 (2H, bt), 7.06 (1H, m), 4.42 (1H, dd, 3.99 (1H, m), 3.83 (1H, m), 3.73 (1H, m), 3.56 (1H, m), 3.38 (8H, m), 3.10 (4H, m), 2.79 (1 H, s), 1.23 (9H, s).

### (+)-4-(4-tert-Butyl-phenyl)-2-[3-fluoro-2-(4-methylpiperazin-1-yl)-benzyl]-morpholin-3-one hydrochloride

White solid. M.P. 104.7° C. dec.

Elemental analysis calculated for C₂₆H₃₄FN₃O₂.HCl.3.5H₂O: C, 57.93, H, 7.85, N, 7.79. Found: C, 58.12, H, 8.35, N, 7.66.

[α]²⁵_{D}=+78.3° (c=0.55, MeOH).

### (−)-4-(4-tert-Butyl-phenyl)-2-[3-fluoro-2-(4-methylpiperazin-1-yl)-benzyl]-morpholin-3-one hydrochloride

White solid. M.P. 128.5-129.9° C.

Elemental analysis calculated for C₂₆H₃₄FN₃O₂.HCl.3H₂O: C, 58.91, H, 7.80, N, 7.93. Found: C, 59.10, H, 7.85, N, 7.66.

[α]²⁵_{D}=−82.4° (c=0.56, MeOH).

### Example 31

### (±)-4-(4-tert-Butyl-phenyl)-2-[5-methyl-2-(4-methylpiperazin-1-yl)-benzyl]-morpholin-3-one (Method A)

Pale yellow oil. Mass spectrum 436 (M+1).

¹H-NMR (CDCl₃, 250 MHz) δ 7.39 (2H, d), 7.23 (2H, d), 7.14 (1H, s), 7.04 (2H, m), 4.68 (1H, dd), 4.11 (2H, m), 3.59 (2H, m), 2.99 (4H, m), 2.87 (2H, m), 2.59 (4H, s), 2.33 (3H, s), 2.28 (3H, s), 1.29 (9H, s).

### (+)-4-(4-tert-Butyl-phenyl)-2-[5-methyl-2-(4-methylpiperazin-1-yl)-benzyl]-morpholin-3-one

Pale yellow solid. M.P. 92-93° C.

Elemental analysis calculated for C₂₇H₃₇N₃O₂: C, 74.48, H, 8.50, N, 9.65. Found: C, 74.31, H, 8.64, N, 9.49.

### (−)-4-(4-tert-Butyl-phenyl)-2-[5-methyl-2-(4-methylpiperazin-1-yl)-benzyl]-morpholin-3-one

Pale yellow solid. M.P. 86-88 “C.

Elemental analysis calculated for C₂₇H₃₇N₃O₂: C, 74.48, H, 8.50, N, 9.65. Found: C, 73.98, H, 8.50, N, 9.50.

### Example 32

### (±)-4-(4-tert-Butyl-phenyl)-2-[2-(4-methylpiperazin-1-yl)-5-trifluoromethyl-benzyl]-morpholin-3-one (Method A)

Off-white solid. M.P. 145.1-146.2° C. Mass spectrum 490 (M+1).

¹H-NMR (CDCl₃, 250 MHz) δ 7.66 (1H, s), 7.50 (1H, dd), 7.39 (2H, d), 7.24 (1H, m), 7.14 (2H, d), 4.46 (1H, dd), 4.10 (1H, m), 3.87 (2H, m), 3.58 (2H, m), 3.20 (6H, m), 3.03 (2H, m), 2.71 (3H, bs), 1.28 (9H, s).

### (+)-4-(4-tert-Butyl-phenyl)-2-[2-(4-methylpiperazin-1-yl)-5-trifluoromethyl-benzyl]-morpholin-3-one hydrochloride

Off-white solid. M.P. 136-138° C.

Elemental analysis calculated for C₂₇H₃₄F₃N₃O₂.HCl.H₂O: C, 59.61, H, 6.86, N, 7.72. Found: C, 59.23, H, 6.74, N, 7.23.

[α]²⁵_{D}=+61.30 (c=0.89, MeOH).

### (−)-4-(4-tert-Butyl-phenyl)-2-[2-(4-methylpiperazin-1-yl)-5-trifluoromethyl-benzyl]-morpholin-3-one hydrochloride

White solid. M.P. 128-130° C.

Elemental analysis calculated for C₂₇H₃₄F₃N₃O₂.HCl.2H₂O: C, 57.70, H, 6.99, N, 7.48. Found: C, 58.12, H, 6.79, N, 7.46.

[α]²⁵_{D}=−69.30 (c=0.76, MeOH).

### Example 33

### (±)-4-(4-tert-Butyl-phenyl)-2-[2-(4-methylpiperazin-1-yl)-4-trifluoromethyl-benzyl]-morpholin-3-one (Method A)

White solid. Mass spectrum 490 (M+1).

¹H-NMR (CDCl₃, 250 MHz) δ 7.47 (1H, d), 7.41 (4H, m), 7.15 (2H, d), 4.40 (1H, m), 3.87 (2H, d), 3.56 (6H, m), 3.21 (1H, dt), 3.11 (3H, m), 2.98 (1H, m), 2.70 (4H, m), 1.55 (2H, bs), 1.29 (9H, s).

### Example 34

### (±)-4-Biphenyl-4-yl-2-[2-(4-methylpiperazin-1-yl)-benzyl]-morpholin-3-one hydrochloride (Method A)

White solid. M.P. 219.4-222.2° C. Mass spectrum 442 (M+1).

¹H-NMR (CD₃OD, 250 MHz) δ 7.66 (2H, dd), 7.59 (2H, d), 7.42 (3H, m), 7.34 (3H, m), 7.25 (2H, m), 7.14 (1H, dt), 4.58 (1H, dd), 4.11 (1H, m), 3.90 (2H, m), 3.65 (1H, m), 3.50 (3H, m), 3.19 (4H, m), 3.06 (1H, bt), 2.92 (3H, s).

### Example 35

### (±)-2-[2-(4-Methylpiperazin-1-yl)-benzyl]-4-pyridin-3-yl-morpholin-3-one hydrochloride (Method A)

White solid. M.P. 175.4° C. dec. Liquefies at 221.6-225.5° C. Mass spectrum 366 (M+1).

¹H-NMR (CD₃OD, 250 MHz) δ 9.27 (1H, d), 8.72 (2H, m), 8.14 (1H, dd), 7.36 (1H, d), 7.23 (2H, m), 7.12 (1H, dt), 4.69 (1H, dd), 4.16 (1H, m), 4.08 (1H, m), 4.00 (1H, dt), 3.82 (1H, m), 3.52 (2H, m), 3.26 (5H, m), 3.17 (2H, m), 3.07 (1H, m), 2.95 (3H, s).

### Example 36

### (±)-4-(4-tert-Butyl-phenyl)-2-[2-(3,4,5-trimethylpiperazin-1-yl)-benzyl]-morpholin-3-one hydrochloride (Method A)

White solid. M.P. 135° C. (dec). Mass spectrum 450 (M+1).

### (+)-4-(4-tert-Butyl-phenyl)-2-[2-(3,4,5-trimethylpiperazin-1-yl)-benzyl]-morpholin-3-one hydrochloride

White solid. M.P. 103.5-105° C.

[α]²⁵_{D}=+69.1° (c=0.52, MeOH).

### (−)-4-(4-tert-Butyl-phenyl)-2-[2-(3,4,5-trimethylpiperazin-1-yl)-benzyl]-morpholin-3-one hydrochloride

White solid. M.P. 103.1-104.8° C.

[α]²⁵_{D}=−78.3° (c=0.59, MeOH).

### Example 37

### (±)-4-(4-tert-Butyl-benzyl)-2-[2-(4-methyl-piperazin-1-yl)-benzyl]-morpholin-3-one hydrochloride (Method A)

White solid. M.P. 215-216° C. Mass spectrum 436 (M+1).

¹H-NMR (DMSO-d₆, 250 MHz) δ 7.32 (2H, d), 7.27 (1H, dd), 7.13 (4H, m), 7.00 (1H, t), 4.45 (1H, d), 3.87 (1H, dt), 3.64 (1H, dq), 3.33 (5H, m), 3.07 (9H, m), 2.77 (3H, s), 2.44 (9H, s).

Elemental analysis calculated for C₂₇H₃₇N₃O₂.HCl.1.5H₂O: C, 64.98, H, 8.28, N, 8.42. Found: C, 64.99, H, 8.28, N, 8.49.

### Example 38

### 2-[2-(4-Methylpiperazin-1-yl)-benzyl]-4-(4-trifluoromethyl-phenyl)-morpholin-3-one hydrochloride (Method C)

Tan solid. Mass spectrum 434 (M+1).

¹H-NMR (DMSO-d₆, 250 MHz) δ 7.78 (2H, dd), 7.66 (2H, dd), 7.35 (1H, d), 7.23 (1H, m), 7.10 (2H, m), 5.74 (2H, bs), 4.65 (1H, d), 4.09 (1H, m), 3.90 (2H, m), 3.72 (1H, m), 3.41 (8H, m), 2.79 (3H, s).

### Example 39

### (−)4-(4-tert-Butyl-phenyl)-2-[2-(4-methyl-4-oxy-piperazin-1-yl)-benzyl]-morpholin-3-one

Under N₂, urea hydrogen peroxide addition complex (0.447 g, 4.72 mmol, 98%, Aldrich Chemical Co.) was added to a stirred solution of (+)-4-(4-tert-butyl-phenyl)-2-[2-(4-methylpiperazin-1-yl)-benzyl]-morpholin-3-one (0.25 g, 0.59 mmol, from Example 24) in 5.0 mL absolute ethanol. This mixture was warmed to 32° C. overnight, then a further six hrs. The reaction was then diluted with water and ethyl acetate, the aqueous layer was further extracted with additional ethyl acetate, and the combined organic layers then washed with water and saturated NaCl solution. The ethyl acetate extracts were finally dried over Na₂SO₄, filtered and concentrated in vacuo to a white foam, 0.213 g. This residue was dissolved in methylene chloride and washed several times with water, dried and concentrated to a white foam, 71 mg. M.P. 89.3-92.8° C.

Elemental analysis calculated for C₂₆H₃₅N₃O₅.2.5H₂O: C, 64.71, H, 8.35, N, 8.71. Found: C, 64.71, H, 8.20, N, 8.50.

(+)4-(4-tert-Butyl-phenyl)-2-[2-(4-methyl-4-oxy-piperazin-1-yl)-benzyl]-morpholin-3-one was prepared in a similar manner from (−)-4-(4-tert-Butyl-phenyl)-2-[2-(4-methylpiperazin-1-yl)-benzyl]-morpholin-3-one to give an off-white foam. M.P. 110.9-115.2° C.

Elemental analysis calculated for C₂₆H₃₅N₃O₅.3H₂O: C, 63.50, H, 8.41, N, 8.55. Found: C, 63.67, H, 8.05, N, 8.55.

### Preparation 1

### 2-(4-Methylpiperazin-1-yl)-benzaldehyde

This compound was prepared using the methods of W. Nijhuis et al, Synthesis, 641-645 (1987) or J. Watthey et al, Journal of Medicinal Chemistry, 26, 1116-1122 (1983).

In a similar manner the following analogs were also prepared:

### 4-Chloro 2-(4-methylpiperazin-1-yl)-benzaldehyde

93% yield as a tan colored oil. Mass spectrum 239 (M⁺¹), 241. ¹H-NMR (CDCl₃, 250 MHz) δ 10.12 (1H, s), 7.7 (1H, d), 7.05 (2H, d), 3.15 (4H, brs), 2.61 (4H, br s), 2.4 (3H, s).

### 6-Fluoro-2-(4-methylpiperazin-1-yl)-benzaldehyde

69% yield as a light brown oil. Mass spectrum 223 (M⁺¹). ¹H-NMR (CDCl₃, 250 MHz) δ 10.27 (1H, s), 7.45 (1H, m), 7.86 (1H, d), 6.75 (1H, dd), 3.14 (4H, t), 2.62 (4H, t), 2.37 (3H, s).

### 3-Fluoro-2-(4-methylpiperazin-1-yl)-benzaldehyde

45% yield as a yellow oil. Mass spectrum 223 (M⁺¹).

### 2-(3,5-Dimethylpiperazin-1-yl)-benzaldehyde

From 2-fluorobenzaldehyde and 2,6-dimethylpiperazine. 36% yield as a light amber colored oil. Mass spectrum 219 (M+1). ¹H-NMR (CDCl₃, 250 MHz) δ 10.25 (1H, s), 7.8 (1H, d), 7.5 (1H, d), 7.05 (2H, dd), 3.15 (4H, m), 2.5 (2H, t), 1.05 (6H, t).

### 2-(3-(R)-Dimethylamino-pyrrolidin-1-yl)-benzaldehyde

Prepared from 2-fluorobenzaldehyde (0.828 g) and (3R)-(+)-dimethylamino)-pyrrolidine (1.1 g), K₂CO₃ (2.3 g), 25 mL H₂O and 2.5 mL 1,4-dioxane at 100° C. for Yield of 1.27 g (87%) as a light amber oil. Mass spectrum 219 (M+1). ¹H-NMR (CDCl₃, 250 MHz) δ 10.05 (1H, s), 7.68 (1H, m), 7.36 (1H, m), 6.80 (2H, m), 3.57 (1H, dq), 3.33 (2H, m), 2.60 (1H, dt), 2.47(1 H, m), 2.27 (6H, s), 2.18 (1H, m), 1.87 (1H, m).

### Preparation 2

### 5-Methyl-2-(4-methylpiperazin-1-yl)-benzaldehyde

This compound was prepared in four steps from commercially available 2-fluoro-5-methylbenzoic acid (Aldrich Chemical Company). Thus, the benzoic acid (3.0 g, 19.5 mmol) in 100 mL of absolute ethanol was treated with acetyl chloride (1.5 g, 19.5 mmole) via syringe at room temperature and after 18 hours was heated to reflux for 5 hours. After cooling to room temperature, concentrated sulfuric acid (0.5 mL) was added and the mixture again heated to reflux for 18 hr. After cooling to room temperature, the volume was reduced to approximately 10 mL in vacuo and then diluted with saturated aqueous NaHCO₃ until the pH was above 7.5 and extracted with ethyl acetate. The organic layers were combined and washed with water and saturated NaCl, dried with MgSO₄ and concentrated to give ethyl 2-fluoro-5-methylbenzoate as a colorless oil, 3.25 g. Mass spectrum 182 (M+).

The above ester (2.2 g, 12.1 mmol) in 15 mL of N-methylpiperazine was heated to reflux under N₂ for 18 hr to produce a dark tan solution. The excess N-methylpiperazine was removed in vacuo and the residue was dissolved in ethyl acetate, then washed with water and saturated NaCl. After drying with MgSO₄, the solvent was removed in vacuo to give a tan oil, 2.49 g. Chromatography on silica gel using 5% methanol in methylene chloride as eluent gave pure ethyl 5-methyl-2-(4-methylpiperazin-1-yl)-benzoate as a yellow oil, 2.04 g. Mass spectrum 263 (M+1).

The preceding ester (1.4 g, 5.34 mmol) in 30 mL of anhydrous THF was cooled to 0° C. and treated with 8.0 mL of 1.0 M LiAlH₄ in THF (8.0 mmol, Aldrich Chemical Co.) via syringe over a 15 min. period. Cooling was removed and the yellow solution stirred for another 3 hr, at which time the reaction was cooled in an ice bath and the mixture quenched with 300 microliters of H₂O, 300 microliters of 15% aqueous NaOH and then 900 microliters of H₂O. After stirring another 1 hr, the mixture was dried with MgSO₄, filtered and concentrated in vacuo to give 5-methyl-2-(4-methylpiperazin-1-yl)-benzyl alcohol as a colorless oil, 1.06 g. Mass spectrum 221 (M+1).

The preceding alcohol (1.0 g, 4.54 mmol) in 25 mL of anhydrous THF was treated with 3.95 g (45.4 mmol) of manganese (IV) oxide. The mixture was stirred at room temperature for 18 hr, then heated to 50° C. for 24 hr, at which time a tic (silica gel, 90 chloroform:10 methanol) showed formation of the less polar product. The mixture was filtered through diatomaceous earth (d.e.) while hot, the pad was washed with additional THF and the solvent was removed in vacuo to give 5-methyl-2-(4-methylpiperazin-1-yl)-benzaldehyde as a yellow oil, 0.808 g. Mass spectrum 219 (M+1). ¹H-NMR (CDCl₃, 400 MHz) δ 10.28 (1H, s), 7.58 (1H, s), 7.30 (1H, m), 7.10 (1H, d), 3.09 (4H, m), 2.66 (4H, bs), 2.39 (3H, s), 2.30 (3H, s).

In a similar manner, 2-fluoro-4-trifluoromethylbenzoic acid was converted to 4-trifluoromethyl-2-(4-methylpiperazin-1-yl)-benzaldehyde. Mass spectrum 273 (M+1). ¹H-NMR (CDCl₃, 400 MHz) δ 10.23 (1H, s), 7.85 (1H, d), 7.34 (1H, dd), 7.29 (1H, s), 3.23 (4H, bs), 2.79 (4H, bs), 2.47 (3H, s).

### Preparation 3

### 2-(3,4,5-Trimethylpiperazin-1-yl)-benzaldehyde

A solution of 2-(3,5-dimethylpiperazin-1-yl)-benzaldehyde (1.2 g, 5.5 mmol), as described in Preparation 1 above, in 11.5 mL THF and 2 mL of H₂O was treated with 0.524 g of formic acid followed by 0.535 g (6.6 mmol) of 37% aqueous formaldehyde and then stirred at room temperature for 48 hr. The mixture was then made basic to pH with aqueous NaHCO₃ and extracted carefully with methylene chloride. The combined organic extractions were washed with water, saturated NaCl and dried with MgSO₄. Concentration in vacuo gave the crude title product as an amber colored oil, 0.696 g. Mass spectrum 233 (M+1). ¹H-NMR (CDCl₃, 400 MHz) δ 10.25 (1H, s), 7.85 (1H, d), 7.5 (1H, m), 7.05 (2H, m), 3.10 (2H, m), 2.85 (2H, bs), 2.60 (2H, bs), 2.35 (3H, bs), 1.12 (6H, m).

### Preparation 4

### 4-Benzyl-morpholin-3-one

Under a nitrogen atmosphere in a flame-dried flask, sodium hydride (120 mg, 3.0 mmol, 60% oil dispersion) was washed with hexanes and then treated with 20 mL of anhydrous DMF, and cooled to 0° C. Morpholin-3-one (253 mg, 2.5 mmol) was added in one portion with stirring. After gas evolution had stopped (ca. 30 min), benzyl chloride (380 mg, 3.0 mmol) was added via syringe and the reaction was stirred at room temperature overnight. The mixture was then treated with 1.0 M HCl and extracted with ethyl acetate. The organic layers were combined, washed with saturated sodium chloride and dried over magnesium sulfate (MgSO₄). Concentration in vacuo gave 672 mg of the title product as a colorless oil. Mass spectrum 191 (M+).

¹H-NMR (CDCl₃, 400 MHz) δ 7.25 (5H, m), 4.50 (2H, s), 4.20 (2H, s), 3.75 (2H, m), 3.23 (2H, m).

In a similar manner, 4-(4-tert-Butylbenzyl)-morpholin-3-one was prepared in 75% yield as a white solid. Mass spectrum 247 (M+). ¹H-NMR (CDCl₃, 400 MHz) δ 7.32 (2H, d), 7.16 (2H, d), 4.56 (2H, s), 4.21 (2H, s), 3.80 (2H, dd), 1.27 (9H, s).

### Preparation 5

### 4-(4-Isopropylphenyl)-morpholin-3-one

This compound was prepared in two steps:

Step A. Under N₂ in a round-bottomed flask with magnetic stirrer and condenser 4-bromo-isopropylbenzene (1.98 g, 10 mmol), morpholine (1.74 g, 20 mmol), toluene (75 mL), palladium acetate (337 mg, 1.5 mmol) and BINAP (934 mg, 1.5 mmol; BINAP=racemic-2,2′-Bis(diphenylphosphino)-1,1′-binaphthyl) were combined and stirred while adding sodium tert-butoxide (3.84 g, 40 mmol). The mixture was heated to reflux overnight, cooled to room temperature and filtered through d.e., washing the filter pad with additional toluene and methylene chloride. The filtrate was concentrated to a black residue, which was chromatographed on silica gel, eluting with chloroform. The product fractions were concentrated in vacuo to give 4-(4-isopropylphenyl)-morpholine as a brown oil which slowly solidified. Yield 0.962 g. Mass spectrum 205 (M+). ¹H-NMR (CDCl₃, 400 MHz) δ 7.13 (2H, d), 6.87 (2H, bs), 3.84 (4H, bs), 3.11 (4H, bs), 2.62 (1H, q), 1.19 (6H, d).

In a similar manner, the following were prepared:

4-(4-tert-butylphenyl)-morpholine. Yellow solid. Mass spectrum 219 (M+). ¹H-NMR (CDCl₃, 400 MHz) δ 7.28 (2H, d), 6.87 (2H, bs), 3.84 (4H, bs), 3.12 (4H, bs), 1.26 (9H, s).

4-(3-trifluoromethylphenyl)-morpholine. Yield 92%. Mass spectrum 231 (M+).

4-(4-trifluoromethylphenyl)-morpholine. Yield 87%. Waxy white solid. Mass spectrum 231 (M+).

4-(3-pyridyl)-morpholine. Yield 85% as an amber oil. Mass spectrum 165 (M+1).

4-(2-pyridyl)-morpholine. Yield 98% as an amber colored oil.

4-(2-pyrimidinyl)-morpholine. Yield 50% as a yellow oil. Mass spectrum 166 (M+1).

4-(4-biphenylyl)-morpholine. White solid. Mass spectrum 240 (M+1).

Step B. Using the method disclosed by J. H. Markgraf and C. A. Stickney (Journal of Heterocyclic Chemistry, 2000, 37(11):109-110), the title compound from step A (0.950 g, 4.63 mmol) in 50 mL of methylene chloride was treated with benzyltriethylammonium chloride (3.15 g, 13.88 mmol) and potassium permanganate (2.19 g, 13.88 mmol), then heated to reflux overnight. After cooling to room temperature, a second portion of benzyltriethylammonium chloride (0.787 g) followed by KMnO₄ (0.548 g) was added and the mixture was again refluxed overnight. On cooling to room temperature, the mixture was poured into 100 mL of water and treated with 20% sodium bisulfite solution while stirring for one hr. The mixture was then filtered through d.e., the pad washed repeatedly with water and methylene chloride, and the organic filtrates were finally washed with water and saturated NaCl. After drying with MgSO₄, the organic solvent was removed in vacuo to give 4-(4-isopropylphenyl)-morpholin-3-one as an orange semisolid, 0.417 g. Mass spectrum 219 (M+). ¹H-NMR (CDCl₃, 400 MHz) δ 7.22 (4H, m), 4.30 (2H, s), 3.98 (2H, m), 3.71 (2H, m), 2.87 (1 H, m), 1.21 (6H, d).

In the same manner, the following morpholin-3-ones were prepared:

4-(4-tert-butylphenyl)-morpholin-3-one. Yield 76% as an orange oil. Mass spectrum 233 (M+). ¹H-NMR (CDCl₃, 400 MHz) δ 7.39 (2H, dd), 7.21 (2H, dd), 4.31 (2H, s), 3.99 (2H, m), 3.71 (2H, m).

4-(4-biphenylyl)-morpholin-3-one. Yield 22% as a light orange solid. Mass spectrum 254 (M+1).

4-(2-pyridyl)-morpholin-3-one. Yield 56% as a white solid. Mass spectrum 179 (M+).

4-(3-pyridyl)-morpholin-3-one. Yield 35% as pale yellow solid. Mass spectrum 179 (M+).

4-phenyl-morpholin-3-one. Yield 45% as a white solid with M.P. 112-113° C. Mass spectrum 178 (M+).

4-(3-trifluoromethylphenyl)-morpholin-3-one. Yield 38% as a yellow oil. Mass spectrum 245 (M+). ¹H-NMR (CDCl₃, 400 MHz) δ 7.58 (1H, s), 7.52 (3H, m), 4.33 (2H, s), 4.03 (2H, m), 3.78 (2H, m).

### Preparation 6

### 4-[4-(1-Hydroxy-1-methylethyl)-phenyl]-morpholin-3-one

To a flame-dried round-bottomed flask under N₂, containing 1.4 M methylmagnesium bromide in toluene (9.0 mL, 12.5 mmol, Aldrich Chemical Co.) and 10 mL THF at 5-10° C., was added a solution of 4-bromobenzophenone (1.99 g, 10 mmol) in 10 mL THF via syringe. The mixture was stirred in the ice bath for 1 hr and then allowed to warm to room temperature while stirring overnight. The mixture was then heated at reflux for 5 hr, after which time the reaction was cooled to room temperature and treated with an additional 9.0 mL of 1.4 M methylmagnesium bromide. The mixture was again refluxed for another 72 hr. The reaction was then cooled to room temperature and quenched with saturated aqueous ammonium chloride, water and ethyl acetate were then added and stirred for 1 hr. The organic layer was separated, washed with water and saturated NaCl, dried with MgSO₄ and concentrated in vacuo to give 2-(4-bromophenyl)-propan-2-ol as a clear colorless oil, 2.09 g. Mass spectrum 216, 218. ¹H-NMR (CDCl₃, 400 MHz) δ 7.38 (2H, d), 7.30 (2H, d), 1.98 (1H, bs).

A mixture of the preceding alcohol (0.9 9, 4.18 mmol), morpholine (0.766g, 8.79 mmol), BINAP (0.393 g, 0.63 mmol) and palladium acetate (0.141 g, 0.63 mmol) in 50 mL toluene was treated with sodium tert-butoxide (1.6 g) and heated to reflux overnight. After cooling to room temperature, the mixture was filtered through a pad of d.e. and the filter pad was washed with additional volumes of ethyl acetate. The combined filtrates were evaporated in vacuo to a black residue which was chromatographed on silica gel, eluting with chloroform, to give crude 2-(4-morpholin-4-ylphenyl)-propan-2-ol as a brown oil. 0.259 g. Mass spectrum 221 (M+).

Under N₂, a mixture of the preceding intermediate (0.25 g, 1.1 mmol), benzyltriethylammonium chloride (0.77 g, 3.39 mol) and potassium permanganate (0.536 g, 3.39 mmol) in 20 mL methylene chloride was heated to reflux for 24 hr. The reaction was then cooled, diluted with 20% aqueous sodium bisulfite and filtered through a pad of d.e., washing with additional water and methylene chloride. The combined organic filtrates were washed with water and saturated NaCl, dried over MgSO₄ and concentrated in vacuo to give crude 4-[4-(1-hydroxy-1-methylethyl)-phenyl]-morpholin-3-one as a brown oil, 0.153 g.

¹H-NMR (CDCl₃, 400 MHz) δ 7.6 (1H, m), 7.5 (1H, m), 7.45 (1H, m), 7.25 (1H, m), 4.25 (2H, s), 4.0 (2H, m), 3.7 (2H, m), 2.2 (1H, bs), 1.5 (6H, s).

1. A compound of the formula I 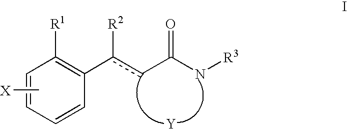
wherein R¹ is a group of the formula G¹, G², G³, G⁴, G⁵, G⁶, G⁷, G⁸ or G⁹ depicted below, 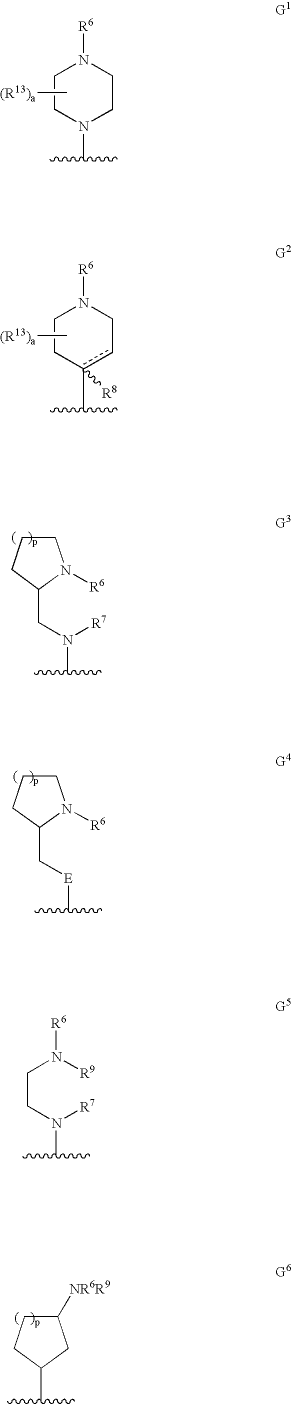 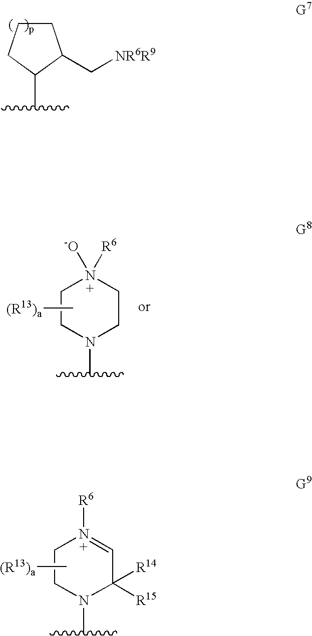
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —SOₜ(C₁-C₆)alkyl wherein t is zero one or two, —CO₂R¹⁰ or —CONR¹¹R¹²;
Y is an optionally substituted (C₁-C₄)heteroalkyl bridge that, together with the atoms to which it is attached, forms a six membered morpholin-3-on-2-yl ring, wherein the substituents on any of the carbon atoms capable of supporting an additional bond are fluoro, (C₁-C₆)alkyl, (C₁-C₆)alkoxy, trifluoromethyl or cyano;
R² is hydrogen, (C₁-C₄)alkyl, phenyl or naphthyl, wherein said phenyl or naphthyl may optionally be substituted with one or more substituents independently selected from chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁶ is selected from the group consisting of hydrogen, (C₁-C₆)alkyl optionally substituted with (C₁-C₆)alkoxy or one to three fluorine atoms, or ((C₁-C₄)alkyl)aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, ((C₁-C₄)alkyl)aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R³ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that may contain from zero to four heteroatoms selected from nitrogen, sulfur and oxygen;
and p is one, two, or three;
each of R¹⁰, R¹¹ and R¹² is selected, independently, from the radicals set forth in the definition of R²; or R¹¹ and R¹², together with the nitrogen to which they are attached, form a 5 to 7 membered heteroalkyl ring that may contain from zero to four heteroatoms selected from nitrogen, sulfur and oxygen; and
the broken lines indicate optional double bonds, with the proviso that when the broken line in G² is a double bond that R⁸ is absent;
or a pharmaceutically acceptable salt thereof.

2. A compound according to claim 1 wherein the R₁ is any of the following groups:

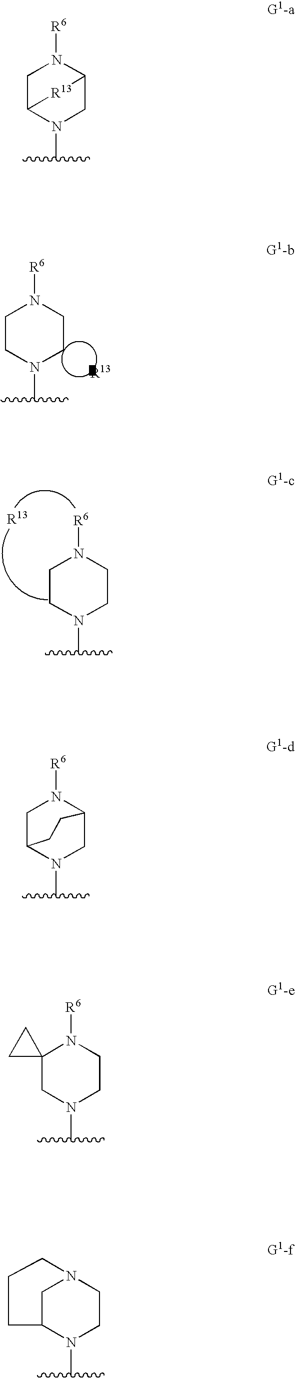

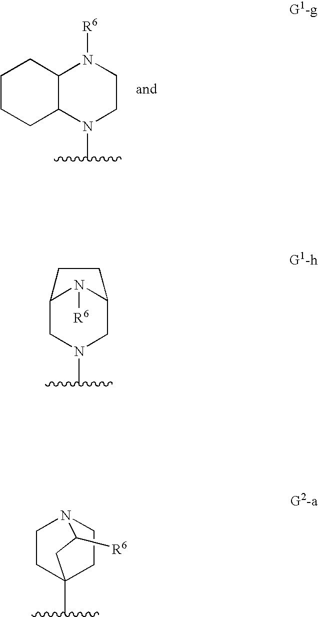

3. A compound according to claim 1, wherein R¹ is 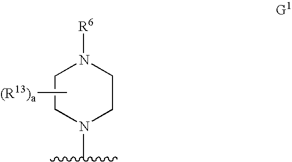
wherein R⁶ is methyl and R¹³ and R² are each hydrogen.

4. A compound according to claim 1 wherein Y, together with the atoms to which it is attached, forms an optionally substituted morpholin-3-on-2-yl.

5. A compound according to claim 1 wherein R³ is optionally substituted phenyl or —(CH₂)-optionally substituted phenyl.

6. A compound according to claim 1, wherein said compound is selected from the group consisting of:
2-[2-(4-Methylpiperazin-1-yl)-benzylidene]-4-(4-isopropylphenyl)-morpholin-3-one,
2-[2-(4-Methylpiperazin-1-yl)-benzylidene]-4-phenyl-morpholin-3-one,
2-[2-(4-Methylpiperazin-1-yl)-benzylidene]-4-(4-tert-butylphenyl)-morpholin-3-one,
2-[2-(3,4,5-Trimethylpiperazin-1-yl)-benzylidene]-4-(4-tert-butylphenyl)-morpholin-3-one,
4-[4-(1-Hydroxy-1-methylethyl)-phenyl]-2-[2-(4-methylpiperazin-1-yl)-benzylidene]-morpholin-3-one,
4-(4-tert-Butyl-phenyl)-2-[4-fluoro-2-(4-methylpiperazin-1-yl)-benzylidene]-morpholin-3-one,
4-(4-tert-Butyl-phenyl)-2-[6-chloro-2-(4-methylpiperazin-1-yl)-benzylidene]-morpholin-3-one,
4-(4-tert-Butyl-phenyl)-2-[4-chloro-2-(4-methylpiperazin-1-yl)-benzylidene]-morpholin-3-one,
4-(4-tert-Butyl-phenyl)-2-[6-fluoro-2-(4-methylpiperazin-1-yl)-benzylidene]-morpholin-3-one,
4-(4-tert-Butyl-phenyl)-2-[3-fluoro-2-(4-methylpiperazin-1-yl)-benzylidene]-morpholin-25 3-one,
4-(4-tert-Butyl-phenyl)-2-[2-(4-methylpiperazin-1-yl)-6-trifluoromethyl-benzylidene]-morpholin-3-one,
4-(4-tert-Butyl-phenyl)-2-[2-(4-methylpiperazin-1-yl)-4-trifluoromethyl-benzylidene]-morpholin-3-one,
4-(4-tert-Butyl-phenyl)-2-[5-methyl-2-(4-methylpiperazin-1-yl)-benzylidene]-morpholin-3-one,
4-(4-tert-Butyl-phenyl)-2-[2-(3-(R)-dimethylamino-pyrrolidin-1-yl)-benzylidene]-morpholin-3-one,
4-(4-tert-Butyl-benzyl)-2-[2-(4-methylpiperazin-1-yl)-benzylidene]-morpholin-3-one,
4-(4-Chlorobenzyl)-5-methyl-2-[2-(4-methylpiperazin-1-yl)-benzylidene]-morpholin-3-one,
(±)-4-(4-tert-Butyl-phenyl)-2-[2-(4-methylpiperazin-1-yl)-benzyl]-morpholin-3-one,
(+)-4-(4-tert-Butyl-phenyl)-2-[2-(4-methylpiperazin-1-yl)-benzyl]-morpholin-3-one,
(−)-4-(4-tert-Butyl-phenyl)-2-[2-(4-methylpiperazin-1-yl)-benzyl]-morpholin-3-one,
(±)-4-(4-Isopropyl-phenyl)-2-[2-(4-methylpiperazin-1-yl)-benzyl]-morpholin-3-one,
(−)-4-(4-Isopropyl-phenyl)-2-[2-(4-methylpiperazin-1-yl)-benzyl]-morpholin-3-one,
(+)-4-(4-Isopropyl-phenyl)-2-[2-(4-methyl piperazin-1-yl)-benzyl]-morpholin-3-one,
(±)-4-(4-tert-Butyl-phenyl)-2-[2-chloro-6-(4-methylpiperazin-1-yl)-benzyl]-morpholin-3-one,
(+)-4-(4-tert-Butyl-phenyl)-2-[2-chloro-6-(4-methylpiperazin-1-yl)-benzyl]-morpholin-3-one,
(−)-4-(4-tert-Butyl-phenyl)-2-[2-chloro-6-(4-methylpiperazin-1-yl)-benzyl]-morpholin-3-one,
(±)-4-(4-tert-Butyl-phenyl)-2-[4-chloro-2-(4-methylpiperazin-1-yl)-benzyl]-morpholin-3-one,
(+)-4-(4-tert-Butyl-phenyl)-2-[4-chloro-2-(4-methylpiperazin-1-yl)-benzyl]-morpholin-3-one,
(−)-4-(4-tert-Butyl-phenyl)-2-[4-chloro-2-(4-methylpiperazin-1-yl)-benzyl]-morpholin-3-one,
(±)-4-(4-tert-Butyl-phenyl)-2-[2-fluoro-6-(4-methylpiperazin-1-yl)-benzyl]-morpholin-3-one,
(+)-4-(4-tert-Butyl-phenyl)-2-[2-fluoro-6-(4-methylpiperazin-1-yl)-benzyl]-morpholin-3-one,
(−)-4-(4-tert-Butyl-phenyl)-2-[2-fluoro-6-(4-methylpiperazin-1-yl)-benzyl]-morpholin-3-one,
(±)-4-(4-tert-Butyl-phenyl)-2-[4-fluoro-2-(4-methylpiperazin-1-yl)-benzyl]-morpholin-3-one,
(+)-4-(4-tert-Butyl-phenyl)-2-[4-fluoro-2-(4-methylpiperazin-1-yl)-benzyl]-morpholin-3-one,
(−)-4-(4-tert-Butyl-phenyl)-2-[4-fluoro-2-(4-methylpiperazin-1-yl)-benzyl]-morpholin-3-one,
(±)-4-(4-tert-Butyl-phenyl)-2-[3-fluoro-2-(4-methylpiperazin-1-yl)-benzyl]-morpholin-3-one,
(+)-4-(4-tert-Butyl-phenyl)-2-[3-fluoro-2-(4-methylpiperazin-1-yl)-benzyl]-morpholin-3-one,
(−)-4-(4-tert-Butyl-phenyl)-2-[3-fluoro-2-(4-methylpiperazin-1-yl)-benzyl]-morpholin-3-one,
(±)-4-(4-tert-Butyl-phenyl)-2-[5-methyl-2-(4-methylpiperazin-1-yl)-benzyl]-morpholin-3-one,
(+)-4-(4-tert-Butyl-phenyl)-2-[5-methyl-2-(4-methylpiperazin-1-yl)-benzyl]-morpholin-3-one,
(−)-4-(4-tert-Butyl-phenyl)-2-[5-methyl-2-(4-methylpiperazin-1-yl)-benzyl]-morpholin-3-one,
(±)-4-(4-tert-Butyl-phenyl)-2-[2-(4-methylpiperazin-1-yl)-5-trifluoromethyl-benzyl]-morpholin-3-one,
(+)-4-(4-tert-Butyl-phenyl)-2-[2-(4-methylpiperazin-1-yl)-5-trifluoromethyl-benzyl]-morpholin-3-one,
(−)-4-(4-tert-Butyl-phenyl)-2-[2-(4-methylpiperazin-1-yl)-5-trifluoromethyl-benzyl]-morpholin-3-one,
(±)-4-Biphenyl-4-yl-2-[2-(4-methylpiperazin-1-yl)-benzyl]-morpholin-3-one,
(±)-4-(4-tert-Butyl-phenyl)-2-[2-(3,4,5-trimethylpiperazin-1-yl)-benzyl]-morpholin-3-one,
(+)-4-(4-tert-Butyl-phenyl)-2-[2-(3,4,5-trimethylpiperazin-1-yl)-benzyl]-morpholin-3-one,
(−)-4-(4-tert-Butyl-phenyl)-2-[2-(3,4,5-trimethylpiperazin-1-yl)-benzyl]-morpholin-3-one,
(±)-4-(4-tert-Butyl-benzyl)-2-[2-(4-methyl-piperazin-1-yl)-benzyl]-morpholin-3-one,
2-[2-(4-Methylpiperazin-1-yl)-benzyl]-4-(4-trifluoromethyl-phenyl)-morpholin-3-one
and the pharmaceutically acceptable salts of such compounds.

7. A compound of the formula V: 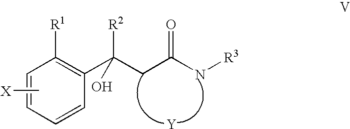
wherein R¹ is a group of the formula G¹, G², G³, G⁴, G⁵, G⁶ or G⁷ depicted below, 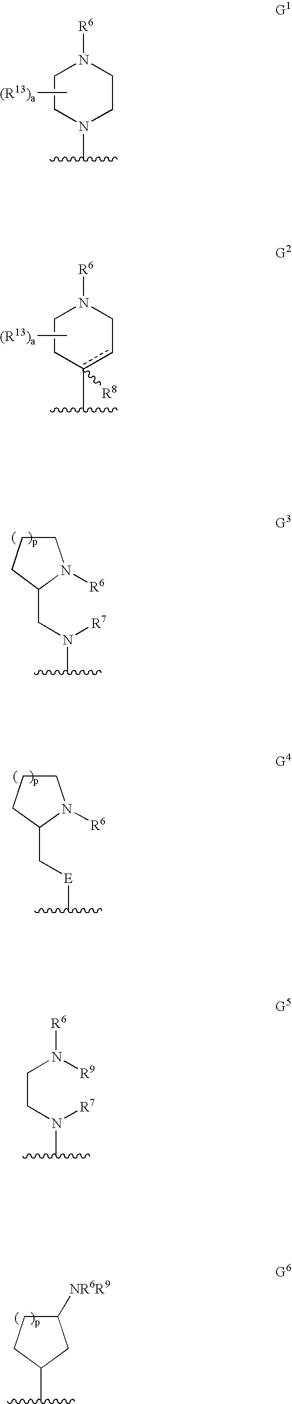 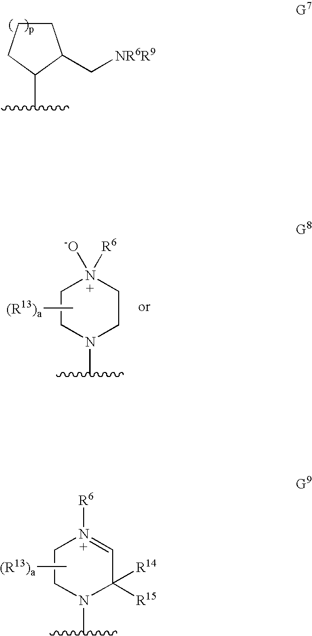
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —SOₜ(C₁-C₆)alkyl wherein t is zero one or two, —CO₂R¹⁰ or —CONR¹¹R¹²;
Y is an optionally substituted (C₁-C₄) heteroalkyl bridge that, together with the atoms to which it is attached, forms a six membered morpholin-3-on-2-yl ring, wherein the substituents on any of the carbon atoms capable of supporting an additional bond, of said (C₁-C₄)heteroalkyl bridge, are fluoro, (C₁-C₆)alkyl, (C₁-C₆)alkoxy, trifluoromethyl or cyano;
R² is hydrogen, (C₁-C₄)alkyl, phenyl or naphthyl, wherein said phenyl or naphthyl may optionally be substituted with one or more substituents independently selected from chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁶ is selected from the group consisting of hydrogen, (C₁-C₆)alkyl optionally substituted with (C₁-C₆)alkoxy or one to three fluorine atoms, or ((C₁-C₄)alkyl)aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, ((C₁-C₄)alkyl)aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that may contain from zero to four heteroatoms selected from nitrogen, sulfur and oxygen;
and p is one, two, or three;
each of R¹⁰, R¹¹ and R¹² is selected, independently, from the radicals set forth in the definition of R²; or R¹¹ and R¹², together with the nitrogen to which they are attached, form a 5 to 7 membered heteroalkyl ring that may contain from zero to four heteroatoms selected from nitrogen, sulfur and oxygen; and
the broken lines indicate optional double bonds, with the proviso that when the broken line in G² is a double bond that R⁸ is absent.

8. A compound according to claim 7 wherein the compound is selected from a group consisting of:
4-(4-tert-Butyl-phenyl)-2-{hydroxy-[2-(4-methylpiperazin-1-yl)-phenyl]-methyl}-morpholin-3-one,
4-(4-tert-Butyl-phenyl)-2-{[4-fluoro-2-(4-methylpiperazin-1-yl)-phenyl]-hydroxymethyl}-morpholin-3-one,
4-(4-tert-Butyl-phenyl)-2-{1-[4-fluoro-2-(4-methylpiperazin-1-yl)-phenyl]-1-hydroxy-ethyl}-morpholin-3-one,
4-[4-(1,1-Dimethylpropyl)-phenyl]-2-{1-hydroxy-1-[2-(4-methylpiperazin-1-yl)-phenyl]-ethyl}-morpholin-3-one,
4-(4-tert-Butyl-phenyl)-2-{[6-fluoro-2-(4-methylpiperazin-1-yl)-phenyl]-hydroxymethyl}-morpholin-3-one,
4-(4-tert-Butyl-phenyl)-2-{[4-chloro-2-(4-methylpiperazin-1-yl)-phenyl]-hydroxymethyl}-morpholin-3-one,
4-(4-Isopropyl-phenyl)-2-{[4-chloro-2-(4-methylpiperazin-1-yl)-phenyl]-hydroxy-methyl}-morpholin-3-one and
4-(4-Isopropyl-phenyl)-2-[2-(4-methylpiperazin-1-yl)-phenyl]-hydroxymethyl-morpholin-3-one.

9. A pharmaceutical composition for treating a disorder or condition that can be treated by enhancing serotonergic neurotransmission in a mammal, preferably a human, comprising (a) an amount of a compound of the formula I, or a pharmaceutically acceptable salt thereof, and (b) a pharmaceutically acceptable carrier that is effective in treating such disorder or condition.

10. A pharmaceutical composition according to claim 9 for treating a disorder or condition selected from the group consisting of: hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, generalized anxiety disorder, phobias, agoraphobia, social phobia, simple phobias, posttraumatic stress syndrome, avoidant personality disorder, premature ejaculation, eating disorders, anorexia nervosa, bulimia nervosa, obesity; chemical dependencies and addictions to alcohol, cocaine, heroin, phenobarbital, nicotine and benzodiazepines; cluster headache, migraine, pain, Alzheimer's disease, obsessive-compulsive disorder, panic disorder, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, bipolar disorder, bipolar disorder-depressed phase, and attention-deficit/hyperactivity disorder (ADHD), in a mammal, preferably a human, comprising (a) an amount of a compound of the formula I or a pharmaceutically acceptable salt thereof and (b) a pharmaceutically acceptable carrier that is effective in treating such disorder or condition.

11. A pharmaceutical composition according to claim 9 for treating a disorder or condition selected from the group consisting of: attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, in a mammal, preferably a human, comprising an amount of (a) a compound of the formula I or a pharmaceutically acceptable salt thereof and (b) a pharmaceutically acceptable carrier that is effective in treating such disorder or condition.

12. A method for treating a disorder or condition that can be treated by enhancing serotonergic neurotransmission in a mammal, preferably a human, comprising administering to a mammal in need of such treatment an amount of a compound of the formula I, or a pharmaceutically acceptable salt thereof that is effective in treating such disorder or condition.

13. A method for treating a disorder or condition according to claim 12 selected from the group consisting of: hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, generalized anxiety disorder, phobias, agoraphobia, social phobia, simple phobias, posttraumatic stress syndrome, avoidant personality disorder, premature ejaculation, eating disorders, anorexia nervosa, bulimia nervosa, obesity; chemical dependencies and addictions to alcohol, cocaine, heroin, phenobarbital, nicotine and benzodiazepines; cluster headache, migraine, pain, Alzheimer's disease, obsessive-compulsive disorder, panic disorder, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, bipolar disorder, bipolar disorder-depressed phase, and attention-deficit/hyperactivity disorder (ADHD), in a mammal, preferably a human, comprising administering to a mammal in need of such treatment an amount of a compound of the formula I, or a pharmaceutically acceptable salt thereof, that is effective in treating such disorder or condition.

14. A method for treating a disorder or condition according to claim 12 selected from attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, in a mammal, preferably a human, comprising administering to a mammal in need of such treatment an amount of a compound of the formula I, or a pharmaceutically acceptable salt thereof, that is effective in treating such disorder or condition.

15. A pharmaceutical composition for treating a disorder or condition that can be treated by enhancing serotonergic neurotransmission in a mammal, preferably a human, comprising a serotonin receptor antagonizing or agonizing effective amount of a compound of the formula I, or a pharmaceutically acceptable salt thereof, and a pharmaceutically acceptable carrier that is effective in treating such disorder or condition.

16. A pharmaceutical composition according to claim 15 for treating a disorder or condition selected from the group consisting of: hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, generalized anxiety disorder, phobias, agoraphobia, social phobia, simple phobias, posttraumatic stress syndrome, avoidant personality disorder, premature ejaculation, eating disorders, anorexia nervosa, bulimia nervosa, obesity; chemical dependencies and addictions to alcohol, cocaine, heroin, phenobarbital, nicotine and benzodiazepines; cluster headache, migraine, pain, Alzheimer's disease, obsessive-compulsive disorder, panic disorder, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, bipolar disorder, bipolar disorder-depressed phase, and attention-deficit/hyperactivity disorder (ADHD), in a mammal, preferably a human, comprising a serotonin receptor antagonizing or agonizing effective amount of a compound of the formula I, or a pharmaceutically acceptable salt thereof, and a pharmaceutically acceptable carrier that is effective in treating such disorder or condition.

17. A pharmaceutical composition for treating a disorder or condition according to claim 15 selected from the group consisting of: attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, in a mammal, preferably a human, comprising a serotonin receptor antagonizing or agonizing effective amount of a compound of the formula I, or a pharmaceutically acceptable salt thereof, and a pharmaceutically acceptable carrier that is effective in treating such disorder or condition.

18. A method for treating a disorder or condition that can be treated by enhancing serotonergic neurotransmission in a mammal, preferably a human, comprising administering to a mammal requiring such treatment a serotonin receptor antagonizing or agonizing effective amount of a compound of the formula I or a pharmaceutically acceptable salt thereof that is effective in treating such disorder or condition.

19. A method for treating a disorder or condition according to claim 18 selected from hypertension, all forms of depression, depression in cancer patients, depression in Parkinson's patients, postmyocardial infarction depression, subsyndromal symptomatic depression, depression in infertile women, pediatric depression, major depressive disorder, single episode depression, recurrent depression, child abuse induced depression, post partum depression, dysthymia; mild, moderate, or severe depressions with or without atypical features, melancholic features, psychotic features, catatonic features; seasonal affective disorder, geriatric depression, chronic depression; adjustment disorder with depressed mood or with anxiety and depressed mood; mixed anxiety and depression; substance induced mood disorder; and mood disorder secondary to a general medical condition, generalized anxiety disorder, phobias, agoraphobia, social phobia, simple phobias, posttraumatic stress syndrome, avoidant personality disorder, premature ejaculation, eating disorders, anorexia nervosa, bulimia nervosa, obesity; chemical dependencies and addictions to alcohol, cocaine, heroin, phenobarbital, nicotine and benzodiazepines; cluster headache, migraine, pain, Alzheimer's disease, obsessive-compulsive disorder, panic disorder, memory disorders, dementia, amnestic disorders, and age-related cognitive decline (ARCD), Parkinson's diseases, dementia in Parkinson's disease, neuroleptic-induced parkinsonism and tardive dyskinesias, endocrine disorders, hyperprolactinaemia, vasospasm, vasospasm in the cerebral vasculature, cerebellar ataxia; gastrointestinal tract disorders involving changes in motility and secretion; negative symptoms of schizophrenia, premenstrual syndrome, fibromyalgia syndrome, stress incontinence, Tourette's syndrome, trichotillomania, kleptomania, male impotence, cancer, small cell lung carcinoma, chronic paroxysmal hemicrania, headache associated with vascular disorders, bipolar disorder, bipolar disorder-depressed phase, and attention-deficit/hyperactivity disorder (ADHD), in a mammal, preferably a human, comprising administering to a mammal requiring such treatment a serotonin receptor antagonizing or agonizing effective amount of a compound of the formula I or a pharmaceutically acceptable salt thereof that is effective in treating such disorder or condition.

20. A method for treating a disorder or condition according to claim 18 selected from the group consisting of: attention-deficit/hyperactivity disorder (ADHD), bipolar disorder, bipolar disorder-depressed phase; mild, moderate, or severe depression with or without atypical features, melancholic features, psychotic features, catatonic features; seasonal affective disorder, postpartum depression, geriatric depression, chronic depression, dysthymia, adjustment disorder with depressed mood, adjustment disorder with anxiety and depressed mood, mixed anxiety and depression, substance induced mood disorder, mood disorder secondary to a general medical condition, in a mammal, preferably a human, comprising administering to a mammal requiring such treatment a serotonin receptor antagonizing or agonizing amount of a compound of the formula I, or a pharmaceutically acceptable salt thereof.
